(19) United States
(12) Patent Application Publication
(10) Pub. No.: US 2010/0298394 A1
(43) Pub. Date: Nov. 25, 2010

(54) ANTIFUNGAL AGENTS AS NEUROPROTECTANTS

(75) Inventors: Joseph P. Steiner, Baltimore, MD (US); Avindra Nath, Ellicott City, MD (US); Norman Haughey, Baltimore, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/815,014

(22) Filed: Jun. 14, 2010

Related U.S. Application Data
(63) Continuation of application No. 12/594,699.
(60) Provisional application No. 60/922,043, filed on Apr. 5, 2007.

(51) Int. Cl. A61K 31/4196; A61K 31/4164; C07D 233/60; C07D 249/08; A61P 25/28; A61P 25/02; A61P 25/04; A61P 25/16; A61P 9/10

(52) U.S. Cl. 514/383; 514/399; 548/342.1; 548/266.6

(57) ABSTRACT

Provided herein are compounds, compositions and methods for protecting neuronal and glial cells.

### CROSS-REFERENCE

This application claims the benefit of U.S. Provisional Application No. 60/922,043, entitled “Role of Antifungal Agents as Neuroprotective Agents,” filed Apr. 5, 2007, which application is incorporated herein by reference.

### STATEMENT AS TO FEDERALLY SPONSORED RESEARCH

This invention was made with the support of the United States government under Contract number P30MH075673 by the NIH/NIMH and R01NS039253 by the NIH/NINDS.

### BACKGROUND OF THE INVENTION

Neurodegenerative conditions such as Alzheimer's Disease, multiple sclerosis, AIDS-related dementia, Huntington's Disease, stroke, and spinal cord trauma are characterized by extensive loss of neurons or glia.

### SUMMARY OF THE INVENTION

Certain antifungal agents, as described in more detail herein, are used for neuroprotection and/or treatment of diseases or conditions associated with neurons or glia. Thus, such antifungal agents are in the form of pharmaceutical compositions, including topical, oral, injectable and intravenous formulations. In addition, such antifungal agents are used in methods for treating diseases or conditions associated with neurons or glia, in which such antifungal agents are adminstered, alone or in combination with other agents, to a patient in need of such treatment. Also, such antifungal agents are used in methods for providing neuroprotection to a patient, in which such antifungal agents are administered to a patient in need of such neuroprotection. Also, described herein are assays and methods for testing the ability of a particular antifungal agent to provide neuroprotection to neurons or glia.

Provided in certain embodiments herein is a method of treating a neurodegenerative disorder, peripheral neuropathy, or neuropathic pain comprising administration to an individual in need thereof a therapeutically effective amount of an antifungal agent. In some embodiments, the neurodegenerative disorder is a neurodegenerative disease selected from, by way of non-limiting example, Alzheimer's Disease, Multiple Sclerosis, HIV-associated dementia, Huntington's Disease, Parkinson's Disease, and Amyotrophic Lateral Sclerosis. In certain embodiments, the neurodegenerative disease is selected from, by way of non-limiting example, Multiple Sclerosis, HIV-associated dementia, Huntington's Disease, Parkinson's Disease, and Amyotrophic Lateral Sclerosis. In some embodiments, the neurodegenerative disorder is a neurodegenerative condition selected from, by way of non-limiting example, stroke and ischemia.

Provided in some embodiments herein is a method of reducing neuronal or glial cell death by contacting a plurality of neurons or glial cells in need of protection from cell death with an effective amount of an antifungal agent. In specific embodiments, the neuronal and/or glial cells are sensory or peripheral; in other embodiments, the neuronal and/or glial cells are neuronal and/or glial cells of the brain and/or spinal cord.

In certain embodiments, at least 5% of the neurons or glial cells are protected from cell death after 18 hours (e.g., as compared to non-treatment or a control composition, such as a placebo or a 0.1% DMSO vehicle).

In some embodiments, the neuronal and/or glial cells protected from cell death by a method described herein are in a patient diagnosed with a neurodegenerative disorder, peripheral neuropathy or neuropathic pain. In certain embodiments, the neurological disorder is selected from, by way of non-limiting example, Alzheimer's Disease, Multiple Sclerosis, HIV-associated dementia, Huntington's Disease, Parkinson's Disease, Amyotrophic Lateral Sclerosis, stroke and ischemia. In some embodiments, the neurological disorder is selected from, by way of non-limiting example, Multiple Sclerosis, HIV-associated dementia, Huntington's Disease, Parkinson's Disease, and Amyotrophic Lateral Sclerosis.

In some embodiments, the antifungal agent utilized in a method described herein is, by way of non-limiting example, an azole antifungal agent. In certain embodiments, the azole antifungal agent is selected from, by way of non-limiting example, an imidazole antifungal agent, a benzimidazole antifungal agent, a triazole antifungal agent, and pharmaceutically acceptable salts thereof. In specific embodiments, the imidazole antifungal agent is selected from, by way of non-limiting example, sulconazole, econazole, clotrimazole, miconazole, bifonazole, fenticonazole, isoconazole, oxiconazole, sertaconazole, tioconazole, and pharmaceutically acceptable salts thereof. In specific embodiments, the imidazole antifungal agent is selected from, by way of non-limiting example, sulconazole, econazole, clotrimazole, miconazole, bifonazole, fenticonizole, isoconazole, oxiconazole, sertaconazole, tioconazole, and pharmaceutically acceptable salts thereof. In some embodiments, the triazole antifungal agent is selected from, by way of non-limiting example, fluconazole, butoconazole, itraconazole, isavuconazole, ravuconazole, posaconazole, voriconazole, albaconazole, terconazole, and pharmaceutically acceptable salts thereof.

In certain embodiments, the antifungal agent utilized in any of the methods described herein is an inhibitor of ergosterol biosynthesis (e.g., fungal). In some embodiments, the antifungal agent is a specific inhibitor of ergosterol biosynthesis. In certain embodiments, the antifungal agent is an inhibitor of lanosterol-14α-demethylase (CYP51). In some embodiments, the antifungal agnet is a specific inhibitor of lanosterol-14α-demethylase (CYP51). In certain embodiments, the inhibitor of lanosterol-14α-demethylase (CYP51) is an azole antifungal agent. In some embodiments, the inhibitor of ergosterol biosynthesis (e.g., fungal) is an inhibitor of squalene epoxidase. In some embodiments, the inhibitor of squalene epoxidase is a thiocarbamate antifungal agent. In specific embodiments, the thiocarbamate antifungal agent is tolnaftate.

In some embodiments, the antifungal agent utilized in any of the methods described herein is an agent that blocks TRPM2 channel signaling.

In certain embodiments, e.g., wherein the method described herein is a method of treating a neurodegenerative disorder or is a method of reducing neuronal or glial cell death by contacting neuronal or glial cells in an individual diagnosed with a neurodegenerative disorder, the antifungal agent is administered systemically. In specific embodiments, the antifungal agent is blood-brain barrier penetrating. In some embodiments, the method is a method of treating peripheral neuropathy or systemic pain and the antifungal agent is administered locally.

In certain embodiments, an antifungal agent, as described herein (including all formulae and specific examples), is combined with an additional agent. One example of an additional agent is an agent that operates via serotonergic mechanisms: an example of such a class of agents are the selective serotonin reuptake inhibitors (SSRIs). Another example of an additional agent is an agent that operates by blocking oxidative stress by neutralizing free radicals and reactive oxygen species: an example of such a class of agents are antioxidants. Another example of an additional agent is an agent that operates by inducing neurotrophic factors such as NGF: an example of such a class of agents are aldose reductase inhibitors.

In one aspect described herein are methods of treating a neurodegenerative disorder, peripheral neuropathy, or neuropathic pain comprising administration to an individual in need thereof a therapeutically effective amount of a compound having the formula:

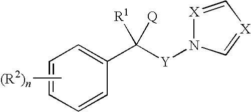

wherein,
each X is independently CH and N, and wherein at least one X is N;
Y is a bond or —CR′R″—;
   R′ and R″ are independently selected from hydrogen, substituted or unsubstituted alkyl, substituted or unsubstituted alkoxy, and halo; or
   R′ and R″, when taken together, are oxo;
Q is H or —C(R³)₃;
n is 5;
R′ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
   R¹ and Q are taken together to form ═N—R⁵, R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl;
R² is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl;
each R³ is independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
   a pair of R³ groups are taken together to form a heterocycloalkyl or cycloalkyl group; or
   three R³ groups are taken together to form a substituted or unsubstituted aryl group or a substituted or unsubstituted heteroaryl group; or
   R⁴ and an R³ taken together are —(C(R⁶)₂)ₘ—;
      each R⁶ is independently selected from hydrogen, halo, -alkyl-O—R⁷, -alkyl-S—R⁷, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl;
      m is 1-5;
         each R⁷ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-aryl-heterocycloalkyl-aryl, and substituted or unsubstituted heteroaryl-aryl-heterocycloalkyl-aryl;
each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl
   or a pharmaceutically acceptable salt thereof.

In some embodiments of the aforementioned methods, the compound is selected from sulconazole, econazole, clotrimazole, miconazole, bifonazole, fenticonizole, isoconazole, oxiconazole, sertaconazole, tioconazole, fluconazole, butoconazole, isavuconazole, ravuconazole, voriconazole, albaconazole, terconazole, posaconazole, or a pharmaceutically acceptable salt, steroisomer, tautomer, or solvate thereof.

In some embodiments of the aforementioned methods, the pharmaceutically acceptable salt thereof is a nitrate.

In some embodiments of the aforementioned methods, the neurodegenerative disorder is a neurodegenerative disease selected from Alzheimer's Disease, Multiple Sclerosis, HIV-associated dementia, Huntington's Disease, Parkinson's Disease, and Amyotrophic Lateral Sclerosis.

In some embodiments of the aforementioned methods, the neurodegenerative disease is selected from Multiple Sclerosis, HIV-associated dementia, Huntington's Disease, Parkinson's Disease, and Amyotrophic Lateral Sclerosis.

In some embodiments of the aforementioned methods, the neurodegenerative disorder is a neurodegenerative condition selected from stroke and ischemia.

In some embodiments of the aforementioned methods, the compound is an inhibitor of fungal ergosterol biosynthesis, including an inhibitor of lanosterol-14α-demethylase (CYP51).

In some embodiments of the aforementioned methods, the method is a method of treating a neurodegenerative condition and the compound is administered systemically.

In some embodiments of the aforementioned methods, the compound is blood-brain barrier penetrating.

In some embodiments of the aforementioned methods, the compound is selected from fluconazole, voriconazole, posaconazole and ravuconazole.

In some embodiments of the aforementioned methods, the method is a method of treating peripheral neuropathy or systemic pain and the compound is administered locally.

In some embodiments of the aforementioned methods, the compound does not inhibit human CYP51.

In some embodiments of the aforementioned methods, the compound blocks TRPM2 channel signaling.

In another aspect are methods of reducing neuronal or glial cell death by contacting a plurality of neurons or glial cells in need of protection from cell death with an effective amount of a compound having the formula:

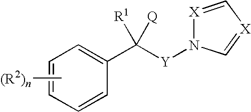

wherein,
each X is independently CH and N, and wherein at least one X is N;
Y is a bond or —CR′R″—;
   R′ and R″ are independently selected from hydrogen, substituted or unsubstituted alkyl, substituted or unsubstituted alkoxy, and halo; or
   R′ and R″, when taken together, are oxo;
Q is H or —C(R³)₃;
n is 5;
R¹ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
   R¹ and Q are taken together to form ═N—R⁵, R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl;
R² is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl;
each R³ is independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
   a pair of R³ groups are taken together to form a heterocycloalkyl or cycloalkyl group; or
   three R³ groups are taken together to form a substituted or unsubstituted aryl group or a substituted or unsubstituted heteroaryl group; or
   R⁴ and an R³ taken together are —(C(R⁶)₂)ₘ—;
      each R⁶ is independently selected from hydrogen, halo, -alkyl-O—R⁷, -alkyl-S—R⁷, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl;
      m is 1-5;
         each R⁷ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-aryl-heterocycloalkyl-aryl, and substituted or unsubstituted heteroaryl-aryl-heterocycloalkyl-aryl;
   each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl
      or a pharmaceutically acceptable salt thereof.

In some embodiments of the aforementioned methods, the neurons are sensory neurons.

In some embodiments of the aforementioned methods, at least 5% of the neurons or glial cells are protected from cell death after 18 hours.

In some embodiments of the aforementioned methods, the plurality of neurons are in a patient diagnosed with a neurodegenerative disorder, peripheral neuropathy or neuropathic pain.

In some embodiments of the aforementioned methods, the neurological disorder is selected from Alzheimer's Disease, Multiple Sclerosis, HIV-associated dementia, Huntington's Disease, Parkinson's Disease, Amyotrophic Lateral Sclerosis, stroke and ischemia.

In some embodiments of the aforementioned methods, the compound is an inhibitor of fungal ergosterol biosynthesis.

In another aspect are pharmaceutical compositions comprising a therapeutically effective amount of a compound having the formula:

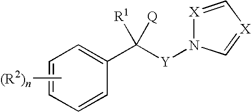

wherein,
each X is independently CH and N, and wherein at least one X is N;
Y is a bond or —CR′R″—;
   R′ and R″ are independently selected from hydrogen, substituted or unsubstituted alkyl, substituted or unsubstituted alkoxy, and halo; or
   R′ and R″, when taken together, are oxo;
Q is H or —C(R³)₃;
n is 5;
R¹ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
   R¹ and Q are taken together to form ═N—R⁵, R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl;
R² is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl;
each R³ is independently selected from hydrogen, halo, —OR⁴, —SR⁴, —N(R⁴)₂, substituted or unsubstituted substituted or unsubstituted aryl, substituted or unsubstituted heteroaryl, substituted or unsubstituted cycloalkyl, substituted or unsubstituted heterocycloalkyl; or
   a pair of R³ groups are taken together to form a heterocycloalkyl or cycloalkyl group; or
   three R³ groups are taken together to form a substituted or unsubstituted aryl group or a substituted or unsubstituted heteroaryl group; or
   R⁴ and an R³ taken together are —(C(R⁶)₂)ₘ—;
      each R⁶ is independently selected from hydrogen, halo, -alkyl-O—R⁷, -alkyl-S—R⁷, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl;
      m is 1-5;
         each R⁷ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-aryl-heterocycloalkyl-aryl, and substituted or unsubstituted heteroaryl-aryl-heterocycloalkyl-aryl;
   each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl
      or a pharmaceutically acceptable salt thereof,
      wherein the therapeutically effective amount is an amount sufficient to reduced neuronal cell death.

In some embodiments of the aforementioned pharmaceutical compositions, the therapeutically effective amount is an amount sufficient to reduce neuronal cell death by at least 5%.

In some embodiments of the aforementioned pharmaceutical compositions, the therapeutically effective amount is an amount sufficient to reduce neuronal cell death by at least 10%.

In some embodiments of the aforementioned pharmaceutical compositions, the composition further comprises a therapeutic agent for treating a neurodegenerative disorder. In some embodiments of the aforementioned pharmaceutical compositions, the compound is fluconazole and the therapeutic agent is paroxetine.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 presents an illustrative titration of 3-NP toxicity in rat mixed hippocampal cultures. Rat mixed hippocampal cultures, day 11-14 in vitro, were subjected to 18 hour exposure to 3-NP, followed by MTT treatment to quantitate cell viability. Based on these data, 3 mM 3-NP, which treatment routinely caused 25-35% decrease in cell viabililty, was used for subsequent assays.

FIG. 2 presents an illustrative validation of 3-NP neurotoxicity assays using known neuroprotective agents, GPI 1046 and the antioxidant resveratrol. Rat mixed hippocampal cultures were pretreated for 1 hour with 10 μM of GPI 1046 and resveratrol, followed by exposure to 3 mM 3-NP for 18 hours. Cell survival was quantitated by MTT endpoint. The statistical significance of data compared to either 3-NP alone is indicated by ANOVA, with Newman-Keuls pairwise post hoc comparisons. Statistical significance ** indicates p<0.01; *** indicates p<0.001.

FIG. 3 presents an illustrative validation of Tat neurotoxicity assays using known neuroprotective agents, GPI 1046 and the antioxidant resveratrol. Rat mixed hippocampal cultures were pretreated for 1 hour with 10 μM of GPI 1046 and resveratrol, followed by exposure to 500 nM Tat for 18 hours. Cell survival was quantitated by MTT endpoint. The statistical significance of data compared to Tat treatment alone is indicated by ANOVA, with Newman-Keuls pairwise post hoc comparisons. Statistical significance ** indicates p<0.01; *** indicates p<0.001.

FIG. 4 presents an illustrative example that certain antifungal agents protect hippocampal neurons from oxidative damage elicited by 3-NP. Mixed hippocampal cultures were treated with increasing concentrations of fluconazole (panel A), miconazole (panel B), clotrimazole (panel C), econazole (panel D), tolnaftate (panel E) and voriconazole (panel F) for 1 h prior to exposure to 3 mM 3-NP for 18 h. Cell survival was measured following 3-NP by MTT assay. Statistical significance was determined by ANOVA with Newman-Keuls pairwise post hoc comparisons. Statistical significance * indicates p<0.05, ** indicates p<0.01; *** indicates p<0.001.

FIG. 5 presents an illustrative example that certain antifungal agents protect hippocampal neurons from HIV-1 Tat toxicity. Mixed hippocampal cultures were treated with increasing concentrations of fluconazole (panel A), miconazole (panel B), clotrimazole (panel C), econazole (panel D), tolnaftate (panel E) and voriconazole (panel F) for 1 h prior to exposure to 500 nM Tat for 18 h. Cell survival was measured following Tat by MTT assay. Statistical significance was determined by ANOVA with Newman-Keuls pairwise post hoc comparisons. Statistical significance * indicates p<0.05, ** indicates p<0.01; *** indicates p<0.001.

FIG. 6 presents an illustrative example that Fluconazole and Voriconazole protect hippocampal neurons from excitotoxic lesions elicited by NMDA. Mixed hippocampal cultures were treated with increasing concentrations of fluconazole (panel A) or voriconazole (panel B), for 1 h prior to exposure to 500 μM N-methyl D-aspartate (NMDA) for 18 h. Cell survival was measured following NMDA by MTT assay. Statistical significance was determined by ANOVA with Newman-Keuls pairwise post hoc comparisons. Statistical significance * indicates p<0.05, ** indicates p<0.01; *** indicates p<0.001.

FIG. 7 presents an illustrative example that Fluconazole and Voriconazole protect hippocampal neurons from 6-OHDA neurotoxicity. Mixed hippocampal cultures were treated with increasing concentrations of fluconazole (panel A) or voriconazole (panel B), for 1 h prior to exposure to 100 μM 6-hydroxydopamine (6-OHDA) for 18 h. Cell survival was measured following 6-OHDA by MTT assay. Statistical significance was determined by ANOVA with Newman-Keuls pairwise post hoc comparisons. Statistical significance * indicates p<0.05, ** indicates p<0.01; *** indicates p<0.00

FIG. 8 presents an illustrative example that Fluconazole and Voriconazole protect hippocampal neurons from toxicity elicited by ADP ribose. Mixed hippocampal cultures were treated with increasing concentrations of fluconazole (panel A) or voriconazole (panel B), for 24 h prior to exposure to 1 mM ADP ribose for 18 h. Cell survival was measured following ADP ribose by MTT assay. Statistical significance was determined by ANOVA with Newman-Keuls pairwise post hoc comparisons. Statistical significance * indicates p<0.05, ** indicates p<0.01; *** indicates p<0.001.

FIG. 9 presents an illustrative example that Fluconazole protects human cortical neurons from 3-NP and 6-OHDA toxicity. Human cortical neuronal cultures were treated with increasing concentrations of fluconazole for 1 h prior to exposure to 3 mM 3-NP (panel A) or 100 μM 6-OHDA (panel B) for 18 h. Cell survival was measured by MTT assay. Statistical significance was determined by ANOVA with Newman-Keuls pairwise post hoc comparisons. Statistical significance * indicates p<0.05, ** indicates p<0.01; *** indicates p<0.001.

FIG. 10 presents an illustrative example that Fluconazole and the SSRI paroxetine synergistically protect rat mixed hippocampal cultures against 3-NP toxicity. Rat mixed hippocampal cultures were treated with 100 nM and 500 nM fluconazole in the presence or absence of 100 nM paroxetine for 1 h prior to exposure to 3 mM 3-NP for 18 h. Cell survival was measured by MTT assay. Statistical significance was determined by ANOVA with Newman-Keuls pairwise post hoc comparisons. Statistical significance with p values is indicated on the figure.

FIG. 11 presents an illustrative example that Fluconazole and the antioxidant N-acetyl cysteine protect rat mixed hippocampal cultures against HIV-1 Tat neurotoxicity. Rat mixed hippocampal cultures were treated with 1 μM and 10 μM N-Acetyl cysteine (NAC) in the presence or absence of 500 nM fluconazole for 1 h prior to exposure to 500 nM HIV-1 Tat for 18 h. Cell survival was measured by MTT assay. Statistical significance was determined by ANOVA with Newman-Keuls pairwise post hoc comparisons. Statistical significance with p values is indicated on the figure.

FIG. 12 presents an illustrative example that Fluconazole and the antioxidant Resveratrol protect rat mixed hippocampal cultures against HIV-1 Tat neurotoxicity. Rat mixed hippocampal cultures were treated with 1 μM and 10 μM Resveratrol (Resv) in the presence or absence of 500 nM fluconazole for 1 h prior to exposure to 500 nM HIV-1 Tat for 18 h. Cell survival was measured by MTT assay. Statistical significance was determined by ANOVA with Newman-Keuls pairwise post hoc comparisons. Statistical significance with p values is indicated on the figure.

FIG. 13 presents an illustrative example that Fluconazole and the aldose reductase inhibitor Sorbinil protect rat mixed hippocampal cultures against HIV-1 Tat neurotoxicity. Rat mixed hippocampal cultures were treated with 100 and 500 nM Fluconazole in the presence or absence of 25 μM Sorbinil for 1 h prior to exposure to 3 mM 3-NP for 18 h. Cell survival was measured by MTT assay. Statistical significance was determined by ANOVA with Newman-Keuls pairwise post hoc comparisons. Statistical significance with p values is indicated on the figure.

### DETAILED DESCRIPTION OF THE INVENTION

Disclosed herein are neuroprotective compounds that decrease induced or spontaneous neuronal or glial cell death, compositions that include the neuroprotective compounds, methods of protecting neuronal or glial cells with neuroprotective compounds disclosed herein from cell death, and methods of using the neuroprotective compounds disclosed herein in treating a neurodegenerative condition. The neuroprotective compounds described herein promote survival of neurons and glia in response to cytotoxic challenges, e.g., oxidative stress. Cytotoxic challenges are associated with a number of neurodegenerative conditions (see, e.g., Lin et al. (2006), Nature, 443(7113):787-795); contact with neurotoxic viral proteins such as HIV Tat (see King et al. (2006), Microbes Infect 2006, 8(5):1347-1357); and hypoxia (see Won et al. (2002), J Biochem Mol Biol 2002, 35(1):67-86). Accordingly, the neuroprotective compounds, compositions, methods described herein can be used to treat a variety of neurodegenerative conditions.

In certain embodiments, provided herein are methods of treating a neurodegenerative disorder, peripheral neuropathy or neuropathic pain comprising administering a therapeutically effective amount of neuroprotective compound, used interchangeably herein with “neuroprotectant”, to an individual in need thereof.

In some embodiments, the methods and compounds described herein are used to treat a chronic neurodegenerative disease, which includes, but is not limited to, Alzheimer's Disease, Multiple Sclerosis, HIV-associated dementia, Schizophrenia, Huntington's Disease, Parkinson's Disease, Amyotrophic Lateral Sclerosis, Multiple System Atrophy, degenerative retinal disease (e.g., macular degeneration), optic neuropathies (e.g., glaucoma, optic nerve stroke, optic neuritis, anterior ischemic optic neuropathy, traumatic optic neuropathy, compressive optic neuropathy, or hereditary neuropathies, such as Leber's hereditary optic neuropathy), Schizophrenia, Pick's disease, Alexander disease, Alper's disease, Ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Kennedy's disease, Krabbe disease, Lewy body dementia, Machado-Joseph disease (Spinocerebellar ataxia type 3), Neuroborreliosis, Pelizaeus-Merzbacher Disease, Primary lateral sclerosis, Prion diseases, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, or any combination thereof.

In some embodiments, the methods and compounds described herein can be used to treat acute neurodegenerative conditions, which include, but are not limited to stroke (e.g., thromboembolic stroke, focal ischemia, global ischemia, or transient ischemic attack), ischemia resulting from a surgical technique involving prolonged halt of blood flow to the brain, head trauma, spinal trauma, or any combination thereof.

Symptoms, diagnostic tests, and prognostic tests for each of the above-mentioned conditions are described in, e.g., the Diagnostic and Statistical Manual of Mental Disorders, 4^{th} ed., 1994, Am. Psych. Assoc.; and Harrison's Principles of Internal Medicine©,” 16th ed., 2004, The McGraw-Hill Companies, Inc.

For example, where the subject is at risk of or is suffering from multiple sclerosis, a set of standard criteria, such as the “McDonald Criteria” can be used for prognosis/diagnosis. See McDonald et al. (2001), Ann Neurol, 50(1):121-127. Magnetic resonance imaging (MRI) of the brain and spine can be used to evaluate individuals with suspected multiple sclerosis. MRI shows areas of demyelination as bright lesions on T2-weighted images or FLAIR (fluid attenuated inversion recovery) sequences. Gadolinium contrast is used to demonstrate active plaques on T1-weighted images. Further, a prognostic biomarker assay of cerebrospinal fluid (CSF) obtained by lumbar puncture can provide evidence of chronic inflammation of the central nervous system. Specifically, CSF is tested for oligoclonal bands, which are immunoglobulins found in 85% to 95% of people with definite MS, albeit not exclusively in MS patients. Additional criteria for diagnosis of multiple sclerosis include, e.g., a reduction in visual evoked potentials and somatosensory evoked potentials, which are indicative of demyelination.

Where a neurodegenerative disorder affects a cognitive ability, a subject can be diagnosed by any one of a number of standardized cognitive assays, e.g., the Mini-Mental State Examination, the Blessed Information Memory Concentration assay, or the Functional Activity Questionnaire. See, e.g., Adelman et al. (2005), Am. Family Physician, 71(9):1745-1750. Indeed, in some cases a subject can also be diagnosed as having a high risk of developing a chronic neurodegenerative condition (e.g., Alzheimer's disease), even in the absence of overt symptoms. For example, the risk of Alzheimer's disease in a subject can be determined by detecting a decrease in the volumes of the subject's hippocampus and amygdala, using magnetic resonance imaging. See, e.g., den Heijer et al. (2006), Arch Gen Psychiatry, 63(1):57-62. Assay of prognostic biomarkers in a sample from a subject are also useful in prognosis or diagnosis of a chronic neurodegenerative condition. For example, where the chronic neurodegenerative condition is Alzheimer's disease, prognostic biomarkers include, but are not limited to, total tau protein, phospho-tau protein, β-amyloid₁₋₄₂ peptide, β-amyloid₁₋₄₀ peptide, complement component 1, q subcomponent (C1q) protein, interleukin 6 (IL-6) protein, apolipoprotein E (APOE) protein, α-1-antichymotrypsin protein, oxysterol (e.g., 24S-hydroxycholesterol), isoprostane (e.g., an F2-isoprostane), 3-nitrotyrosine, homocysteine, or cholesterol, or any combination thereof, e.g., the ratio of β-amyloid₁₋₄₂ peptide to β-amyloid₁₋₄₀ peptide.

The type of biological sample utilized in prognostic Alzheimer's biomarker assays will vary depending on the prognostic biomarker to be measured. Further, the relationship between the level of a prognostic biomarker and Alzheimer's risk varies depending on the particular biomarker, as well as on the biological sample in which the level of the biomarker is determined. In other words, the level of the biomarker in a biological sample may be directly correlated or inversely correlated with the risk of Alzheimer's Disease, as summarized in Table 1.

TABLE 1
ALZHEIMER'S DISEASE PROGNOSTIC BIOMARKERS

| | Biological | Correlation to | |
|---|---|---|---|
| Biomarker | Sample Type | Dementia Risk | Reference |
| tau protein | cerebrospinal | increased | Hampel et al. (2004), Mol Psychiatry, 9: 705-710 |
| | fluid (CSF) | | |
| phospho-tau protein | CSF | increased | Hampel et al. (2004), Arch Gen Psychiatry, 61: 95-102 |
| | | | Hansson et al. (2006), Lancet Neural, 5(3): 228-234 |
| β-amyloid₁₋₄₂ peptide | CSF | decreased | Hampel et al. (2004), Mol Psychiatry, 9: 705-710 |
| Ratio of β-amyloid₁₋₄₂ | plasma | decreased | Graff-Radford et al. (2007), Arch Neurol, 64(3): 354- |
| peptide to β-amyloid₁₋₄₀ | | | 362; |
| peptide | CSF | decreased | Hansson et al. (2007), Dement Geriatr Cogn Disord, |
| | | | 23(5): 316-20 |
| C1q protein | CSF | decreased | Smyth et al. (1994), Neurobiol Aging, 15(5): 609-614 |
| IL-6 protein | plasma | increased | Licastro et al. (2000), J Neuroimmunol, 103: 97-102; |
| | CSF | increased | Sun et al. (2003), Dement Geriatr Cogn Disord, |
| | | | 16(3): 136-44 |
| APOE protein | CSF | increased | Fukuyama et al. (2000), Eur Neurol, 43(3): 161-169 |
| α-1-antichymotrypsin | plasma | increased | Dik et al. (2005), Neurology, 64(8): 1371-1377. |
| protein | | | |
| oxysterol | CSF | increased | Papassotiropoulos et al. (2002), J Psychiatr Res, |
| | | | 36(1): 27-32 |
| isoprostane | CSF | increased | Montine et al. (2005), Antioxid Redox Signal, |
| | | | 7(1-2): 269-275 |
| 3-nitrotyrosine | CSF | increased | Tohgi et al. (1999), Neurosci Lett, 269(1): 52-54 |
| homocysteine | plasma | increased | Seshadri et al. (2002), N Engl J Med, 346(7): 476-83 |
| cholesterol | plasma | increased | Panza et al. (2006), Neurobiol Aging, 27(7): 933-940 |

Animal models are useful for establishing a range of therapeutically effective doses of neuroprotective compounds for treating any of the foregoing diseases. For example, animal models of chronic neurodegenerative conditions have been established, e.g., for Alzheimer's disease, multiple sclerosis, amyotrophic lateral sclerosis, multiple system atrophy, and Huntington's disease. See, e.g., Spires et al. (2005), NeuroRx., 2(3):447-464; Gold et al. (2006), Brain, 129(8):1953-1971; Wong et al. (2002), Nat. Neurosci., 5(7):633-639, Stefanova et al. (2005), Am J Pathol, 166(3): 869-876, Tadros et al. (2005), Pharmacol Biochem Behav; 82(3):574-582. These animal models develop a chronic neurodegenerative condition that is manifested behaviorally by impaired learning, memory, or locomotion. Cognitive abilities, as well as motor functions in non-human animals suffering from a chronic neurodegenerative condition can be assessed using a number of behavioral tasks. Well-established sensitive learning and memory assays include the Morris Water Maze (MWM), context-dependent fear conditioning, cued-fear conditioning, and context-dependent discrimination. See, e.g., Anger (1991), Neurotoxicology, 12(3):403-413. Locomotor behavior, e.g., following spinal trauma, is commonly assessed using a 21-point open field locomotion score assay developed by Basso, Beattie, and Bresnahan (BBB) (Basso, et al. (1995), J. Neurotrauma, 12(1): 1-21). Such animal models are suitable for testing effective dose ranges for the neuroprotective compounds and compositions described herein as well as for identifying additional neuroprotective compounds.

Provided in certain embodiments herein is method of reducing neuronal or glial cell death by contacting a plurality of neuronal and/or glial cells with an effective amount of any neuroprotective compound described herein. In certain embodiments, the neuronal and/or glial cells are in need of protection, e.g., are subject to a stressor and/or toxin, such as, by way of non-limiting example, oxidative stress, a excitatory stress, and/or neurotoxic stress. In specific embodiments, the neuronal and/or glial cells are protected from cell death after 18 hours of being subject to a stressor as compared to treatment with a control agent (e.g., a placebo or a 0.1% DMSO composition) or to non-treatment. In more specific embodiments, at least 5%, at least 10%, at least 15%, at least 20%, at least 30%, at least 40%, at least 50%, at least 60%, at least 70%, at least 80%, at least 90%, or at least 100%. In certain embodiments, protection of neuronal and/or glial cells is measured according to an assay provided herein.

In some embodiments, the neuronal and/or glial cells protected are present in a patient diagnosed with a neurodegenerative disorder, peripheral neuropathy or neuropathic pain. In specific embodiments, the neurological disorder is selected from Alzheimer's Disease, Multiple Sclerosis, HIV-associated dementia, Huntington's Disease, Parkinson's Disease, Amyotrophic Lateral Sclerosis, stroke and ischemia. In some embodiments, the neurons are sensory neurons.

### Certain Chemical Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, methods of mass spectroscopy, NMR, HPLC, protein chemistry, biochemistry, recombinant DNA techniques and pharmacology are employed. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection).

As used herein, C₁-Cₓ includes C₁-C₂, C₁-C₃ . . . C₁-Cₓ.

An “alkyl” group refers to a straight-chained, branched or cyclic aliphatic hydrocarbon group. The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, cyclopropyl, cyclobutyl, cyclopentyl, cyclohexyl, and the like. Alkyl groups include those that are saturated and those that are unsaturated, e.g., alkyl, alkenyl and alkynyl.

An “alkoxy” group refers to a (alkyl)O— group, where alkyl is as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides can be found in Greene and Wuts, Protective Groups in Organic Synthesis, 3^{rd} Ed., John Wiley & Sons, New York, N.Y., 1999, which is incorporated herein by reference in its entirety.

The term “bond” or “single bond” refers to a chemical bond between two atoms, or two moieties when the atoms joined by the bond are considered to be part of larger substructure.

The term “carbocyclic” or “carbocycle” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. Carbocycles include cycloalkyls and aryls.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

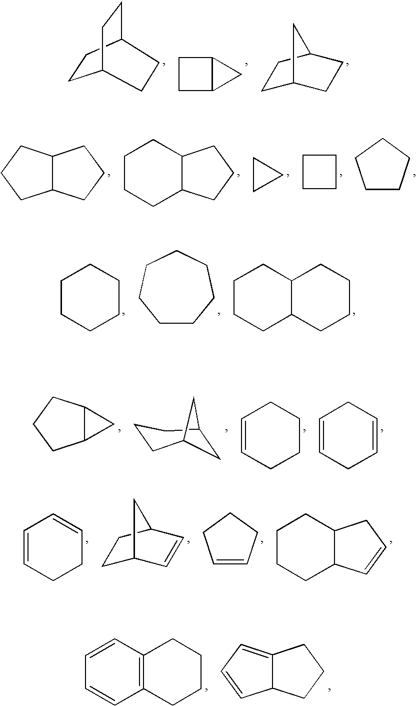

and the like. Depending on the structure, an cycloalkyl group can be a monoradical or a diradical (e.g., an cycloalkylene group). In a heterocycloalkyl, at least one of the carbons of a cycloalkyl is replaced with a heteroatom, e.g., O, S, or N.

“Cycloalkylalkyl” means an alkyl radical, as defined herein, substituted with a cycloalkyl group. Non-limiting cycloalkylalkyl groups include cyclopropylmethyl, cyclobutylmethyl, cyclopentylethyl, cyclohexylmethyl, and the like.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters can be found in Greene and Wuts, Protective Groups in Organic Synthesis, 3^{rd} Ed., John Wiley & Sons, New York, NY, 1999, which is incorporated herein by reference in its entirety.

The term “halo” or, alternatively, “halogen” or “halide” means fluoro, chloro, bromo or iodo.

The term “haloalkyl” includes alkyl, alkenyl and alkynyl structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the term “heteroalkyl” include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

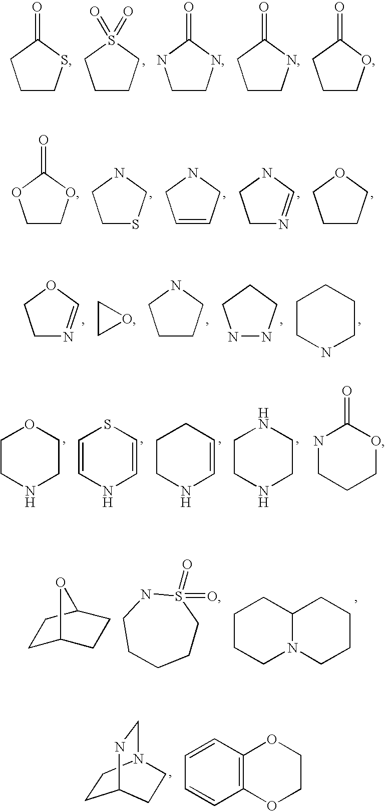

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heterocycloalkylalkyl” refers to an alkyl group, as defined herein, substituted with a heterocycloalkyl, as defined herein.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be Optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group). Phosphorous-containing rings include, but are not limited to, 1-oxo-phospholanyl, 1-methyl-1-oxo-phosphinan-4-yl, 1-phenyl-1-oxo-phosphinan-4-yl, 1-(cyclopropylmethyl)-1-oxo-phosphinan-4-yl, 4-methyl-4-oxo-[1,4]azaphosphiran-1-yl, 4-phenyl-4-oxo-[1,4]azaphosphinan-1-yl, and 4-(cyclopropylmethyl)-4-oxo-[1,4]azaphosphinan-1-yl.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “moiety” refers to a specific segment or functional group of a molecule. Chemical moieties are often recognized chemical entities embedded in or appended to a molecule.

As used herein, the term “O-carboxy” or “acyloxy” refers to a group of formula RC(═O)O—.

“Alkylcarbonyloxy” refers to a (alkyl)-C(═O)O— group.

As used herein, the term “alkoxycarbonyl” refers to a group of formula —C(═O)OR.

“Carboxy” means a —C(O)OH radical.

As used herein, the term “acetyl” refers to a group of formula —C(═O)CH₃.

“Acyl” refers to the group —C(O)R, wherein R is an alkyl group.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, carbonyl, isocyanato, thiocyanato, isothiocyanato, nitro, perhaloalkyl, fluoroalkyl, silyl, and amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers, if desired, may be obtained, for example, by the separation of stereoisomers by chiral chromatographic columns.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein. In certain embodiments, pharmaceutically acceptable salts include, by way of non-limiting example, nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-tolunenesulfonate, mesylate and the like.

### Neuroprotective Compounds

The neuroprotective compounds for use in the pharmaceutical compositions and methods described herein are antifungal agents. In certain embodiments, provided herein is a method of treating a neurodegenerative disorder, peripheral neuropathy or neuropathic pain comprising administering a therapeutically effective amount of neuroprotective compound, used interchangeably herein with “neuroprotectant”, to an individual in need thereof.

In certain embodiments, the antifungal agent is selected from an azole, a thiocarbamate, and an allylamine antifungal agent. In some embodiments, the azole antifungal agent is selected from an imidazole antifungal agent, a benzimidazole antifungal agent, a triazole antifungal agent. In specific embodiments, imidazole antifungal agents are selected from, by way of non-limiting example, sulconazole, econazole, clotrimazole, miconazole, bifonazole, fenticonizole, isoconazole, oxiconazole, sertaconazole, tioconazole, and pharmaceutically acceptable salts thereof. In other specific embodiments, triazole antifungal agents are selected from, by way of non-limiting example, fluconazole, itraconazole, isavuconazole, ravuconazole, posaconazole, voriconazole, terconazole, and pharmaceutically acceptable salts thereof.

In some embodiments, the neuroprotective compound or antifungal agent is an inhibitor of sterol biosynthesis. In certain embodiments, the inhibitor of sterol biosynthesis is an inhibitor of ergosterol biosynthesis. In certain embodiments, the inhibitor of sterol and/or ergosterol biosynthesis is an inhibitor of lanosterol-14α-demethylase (CYP51), which is a member of the cytochrome P450 monooxygenase family. In some embodiments, the inhibitor of lanosterol-14α-demethylase (CYP51) is a selective inhibitor of lanosterol-14a-demethylase (CYP51). In certain embodiments, an inhibitor or selective inhibitor of lanosterol-14α-demethylase (CYP51) does not substantially inhibit the synthesis of cortisol. In certain embodiments, the inhibitor of CYP51 is an azole antifungal agent, which are described herein. Cytochrome P450 isoenzyme 51 (CYP51, lanosterol-14α-demethylase) is a family of highly conserved monooxygenases found in mycobacteria, fungi, plants, animals and humans. In mammals and yeasts, it catalyzes the oxidative removal of lanosterol's 32-methyl group to produce follicular fluid-meiosis activating steroid (FF-MAS), which is an important step in sterol biosynthesis. In fungi, additional catalytic steps result in the generation of ergosterol, which is an essential compound of the fungal cell membrane. In certain instances, inhibitors of sterol and/or ergosterol biosynthesis (e.g., azole antifungal agents) acting as antifungal agents selectively inhibit CYP51, causing fungi to lack ergosterol, which leads to a collapse of the cell membrane.

In certain embodiments, a neuroprotective compound or antifungal agent disclosed herein is an inhibitor of lanosterol-14α-demethylase (CYP51), 11β-hydroxylase or a combination thereof. In some embodiments, a neuroprotective compound or antifungal agent disclosed herein is an inhibitor of either of lanosterol-14α-demethylase (CYP51) or 11β-hydroxylase, but not both. In certain embodiments, inhibition of one of lanosterol-14α-demethylase (CYP51) or 11β-hydroxylase includes the inhibition of one over the other by 5×, 10×, 20×, 30×, 40×, 50×, 75×, 100×, or 1000×.

In animals and humans, cholesterol is a downstream product of lanosterol-14α-demethylation. Cholesterol is necessary for the synthesis of bile acids, mineralocorticoids, glucocorticoids and sex steroids. Inhibition of CYP51 results in a lack of FF-MAS and its following metabolite testis-meiosis activating steroid (T-MAS). These direct products of the CYP51 reaction act as meiosis-activating steroids on ovaries and testes.

Thus, in specific embodiments, the inhibitor of CYP51 inhibits fungal CYP51 at least 10×, 20×, 30×, 50×, 100×, 200×, 300×, or 400× more than human CYP51. A number of compounds have relatively small differences (less than 10 fold) in inhibition of human and fungal CYP51, while some agents, such as, by way of non-limiting example, fluconazole and itraconazole have potencies of greater than 400 fold for fungal CYP51 versus human CYP51. In further or alternative embodiments, embodiments, a CYP51 inhibitor is combined with cholesterol in a composition or methods described herein.

In some embodiments, the inhibitor of sterol and/or ergosterol is an inhibitor of squalene epoxidase. In specific embodiments, the inhibitor of squalene epoxidase is selected from, by way of non-limiting example, a thiocarbamate and an allylamine antifungal agent.

In some embodiments, the antifungal agent utilized in the methods and compositions described herein is selected from, by way of non-limiting example, clotrimazole, exalamide, griseofulvin, fluconazole, sulconazole, flutrimazole, tolnaftate, econazole, triacetin, miconazole, rhapontin, nystatin, pharmaceutical acceptable salts and/or pharmaceutically acceptable derivatives and analogs thereof. In certain embodiments, the antifungal agent is not resveratrol. In some embodiments, the antifungal agent is not ketoconazole.

In certain embodiments, the antifungal agent is an agent that blocks the toxic effects of oxidative stress-induced and/or injury promoted increases in ADP ribose. In certain embodiments, the antifungal agent is an agent that activates transient receptor potential (TRP) M2 channels. In some embodiments, the antifungal agent is an agent that blocks the toxic effects of oxidative stress-induced and/or injury promoted increases in ADP ribose, which in turn activates transient receptor potential (TRP) M2 channels.

In some embodiments, the antifungal agent is suitable for systemic administration and/or is administered systemically. In certain embodiments, the antifungal agent is capable of penetrating the blood-brain barrier. In specific embodiments, at least 20%, at least 30%, at least 40%, at least 50%, at least 60%, at least 70%, at least 80%, or at least 90% of the administered dose of antifungal agent crosses or is capable of crossing the blood-brain barrier. In more specific embodiments, the antifungal agent that crosses or is capable of crossing the blood-brain barrier is, by way of non-limiting example, fluconazole, voriconazole, posaconazole or ravuconazole. In some embodiments, the antifungal agent is suitable for topical or local administration and/or is administered topically or locally.

In certain embodiments, the neuroprotectant utilized in the methods and compositions described herein is selected from, by way of non-limiting example, sulconazole, econazole, clotrimazole, miconazole, bifonazole, fenticonazole, isoconazole, oxiconazole, sertaconazole, tioconazole, fluconazole, butoconazole, itraconazole, isavuconazole, ravuconazole, posaconazole, voriconazole, albaconazole, terconazole, pharmaceutical acceptable salts and/or pharmaceutically acceptable derivatives and analogs thereof. In certain embodiments, the derivatives and analogs of the recited neuroprotectants are derivatives and analogs that retain at least 20%, at least 30%, at least 40%, at least 50%, at least 60%, at least 70%, at least 80% or at least 90% of the neuroprotective characteristic of their respective patent neuroprotectant.

In specific embodiments, the neuroprotectant is tolnaflate, or pharmaceutically acceptable salts, analogs and/or derivatives thereof. In other specific embodiments, the neuroprotectant is econazole, or pharmaceutically acceptable salts, analogs and/or derivatives thereof. In a more specific embodiments, the neuroprotectant is econazole nitrate. In other specific embodiments, the neuroprotectant is fluconazole, or pharmaceutically acceptable salts, analogs and/or derivatives thereof.

In certain embodiments, the neuroprotectant described herein is a compound of Formula I, or a pharmaceutically acceptable salt, steroisomer, tautomer, metabolite or solvate thereof:

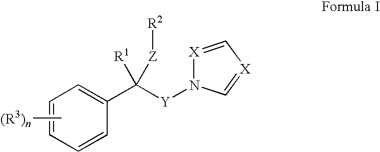

In some embodiments, each X is independently selected from CH and N, and wherein at least one X is N. In certain embodiments, the term Y is a bond or —CR′R″—, wherein the terms R′ and R″ are independently selected from hydrogen, substituted or unsubstituted alkyl, substituted or unsubstituted alkoxy, and halo, or wherein the terms R′ and R″, when taken together, are oxo. In some embodiments, the term Z is a bond, —S—, substituted or unsubstituted —S-alkyl-, substituted or unsubstituted -alkyl-S—, —O—, substituted or unsubstituted —O-alkyl-, substituted or unsubstituted -alkyl-O—, substituted or unsubstituted -aryl-, substituted or unsubstituted -alkyl-, substituted or unsubstituted -alkyl-aryl-, substituted or unsubstituted -aryl-alkyl-, substituted or unsubstituted -cycloalkyl-alkyl-, and substituted or unsubstituted -alkyl-cycloalkyl-. In certain embodiments, the terms R¹, R² and R³ are independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl. In some embodiments, n is 0-5. In certain embodiments, each R⁴ is independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl. In some embodiments, R¹ and R² are selected from —OR⁴, —SR⁴, and —N(R⁴)₂ and a pair of R⁴ groups or an R⁴ group and R¹ or R² are, taken together, —(C(R⁵)₂)ₘ—, forming a heterocycle. In certain embodiments, each R⁵ is independently selected from hydrogen, halo, -alkyl-O—R⁶, -alkyl-S—R⁶, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl. In some embodiments, m is 1-5. In certain embodiments, each R⁶ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, substituted or unsubstituted heterocycloalkyl-aryl, substituted or unsubstituted aryl-heterocycloalkyl, substituted or unsubstituted heterocycloalkyl-aryl-heterocycloalkyl-aryl, and substituted or unsubstituted heteroaryl-aryl-heterocycloalkyl-aryl. In specific embodiments, any of the groups described herein as being substituted are substituted with 1-5 substituents. In more specific embodiments, the substituents are selected from hydrogen, halo, hydroxy, thioxy, amino, amides, acetyl, alkoxy, alkylthioxy, cyano, alkyl, hydroxyalkyl, aryl, heteroaryl, cycloalkyl, and heterocycloalkyl.

In some embodiments, disclosed herein is a compound of Formula I, wherein each X is independently selected from CH and N, and wherein at least one X is N. In certain embodiments, the term Y is a bond or —CR′R″—, wherein the terms R′ and R″ are independently selected from hydrogen, substituted or unsubstituted alkyl, substituted or unsubstituted alkoxy, and halo, or wherein the terms R′ and R″, when taken together, are oxo. In some embodiments, the term Z is a bond, —S—, substituted or unsubstituted —S-alkyl-, substituted or unsubstituted -alkyl-S—, —O—, substituted or unsubstituted —O-alkyl-, substituted or unsubstituted -alkyl-O—, substituted or unsubstituted -aryl-, substituted or unsubstituted -alkyl-, substituted or unsubstituted -alkyl-aryl-, substituted or unsubstituted -aryl-alkyl-, substituted or unsubstituted -cycloalkyl-alkyl-, and substituted or unsubstituted -alkyl-cycloalkyl-. In certain embodiments, the terms R¹, R² and R³ are independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl. In some embodiments, n is 0-5. In certain embodiments, each R⁴ is independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl. In some embodiments, R¹ and R² are selected from —OR⁴, —SR⁴, and —N(R⁴)₂ and a pair of R⁴ groups or an R⁴ group and R¹ or R² are, taken together, —(C(R⁵)₂)ₘ—, forming a heterocycle. In certain embodiments, each R⁵ is independently selected from hydrogen, halo, -alkyl-O—R⁶, -alkyl-S—R⁶, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl. In some embodiments, m is 1-5. In certain embodiments, each R⁶ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted aryl-heteroaryl-, substituted or unsubstituted heteroaryl-aryl-, and substituted or unsubstituted aryl-heterocycloalkyl-. In specific embodiments, any of the groups described herein as being substituted are substituted with 1-5 substituents. In more specific embodiments, the substituents are selected from hydrogen, halo, hydroxy, thioxy, amino, amides, acetyl, alkoxy, alkylthioxy, cyano, alkyl, hydroxyalkyl, aryl, heteroaryl, cycloalkyl, and heterocycloalkyl.

In certain embodiments, disclosed herein is a compound of Formula I, wherein each X is independently selected from CH and N, and wherein at least one X is N. In certain embodiments, the term Y is a bond or —CR′R″—, wherein the terms R′ and R″ are independently selected from hydrogen, substituted or unsubstituted alkyl, substituted or unsubstituted alkoxy, and halo, or wherein the terms R′ and R″, when taken together, are oxo. In some embodiments, the term Z is a bond, —S—, substituted or unsubstituted —S-alkyl-, substituted or unsubstituted -alkyl-S—, —O—, substituted or unsubstituted —O-alkyl-, substituted or unsubstituted -alkyl-O—, substituted or unsubstituted -aryl-, substituted or unsubstituted -alkyl-, substituted or unsubstituted -alkyl-aryl-, substituted or unsubstituted -aryl-alkyl-, substituted or unsubstituted -cycloalkyl-alkyl-, and substituted or unsubstituted -alkyl-cycloalkyl-. In certain embodiments, the term R′ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl. In some embodiments, R² is selected from hydrogen, halo, substituted or unsubstituted alkyl, substituted or unsubstituted aryl, substituted or unsubstituted heteroaryl, substituted or unsubstituted cycloalkyl, substituted or unsubstituted heterocycloalkyl. In some embodiments, each R³ is independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl. In some embodiments, n is 0-5. In certain embodiments, each R⁴ is independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl. In specific embodiments, any of the groups described herein as being substituted are substituted with 1-5 substituents. In more specific embodiments, the substituents are selected from hydrogen, halo, hydroxy, thioxy, amino, amides, acetyl, alkoxy, alkylthioxy, cyano, alkyl, hydroxyalkyl, aryl, heteroaryl, cycloalkyl, and heterocycloalkyl.

In specific embodiments; the compound of Formula I is selected from sulconazole, clotrimazole, fluconazole, sulconazole, flutrimazole, econazole, miconazole, and pharmaceutically acceptable salts thereof. In a more specific embodiment, the pharmaceutically acceptable salt thereof is a nitrate thereof.

In some embodiments, the neuroprotectant described herein is a compound of Formula II, or a pharmaceutically acceptable salt, steroisomer, tautomer, metabolite or solvate thereof:

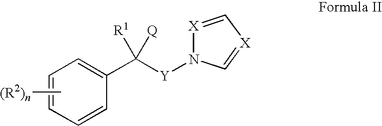

In some embodiments, each term X is independently CH or N, and wherein at least one X is N. In certain embodiments, the term Y is a bond or —CR′R″—, wherein the terms R′ and R″ are independently selected from hydrogen, substituted or unsubstituted alkyl, substituted or unsubstituted alkoxy, and halo, or wherein the terms R′ and R″, when taken together, are oxo. In some embodiments, the term Q is H or —C(R³)₃. In certain embodiments, the terms R¹, R² and R³ are independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl. In some embodiments, R¹ and Q are taken together to form ═N—R⁵, wherein R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl. In certain embodiments, a pair of R³ groups are taken together to form a heterocycloalkyl or cycloalkyl group. In some embodiments, three R³ groups are taken together to form a substituted or unsubstituted aryl group or a substituted or unsubstituted heteroaryl group. In some embodiments, n is 0-5. In some embodiments, R⁴ and an R³ taken together are —(C(R⁶)₂)ₘ—, forming a heterocycle. In certain embodiments, each R⁶ is independently selected from hydrogen, halo, -alkyl-O—R⁷, -alkyl-S—R⁷, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl. In some embodiments, m is 1-5. In certain embodiments, each R⁷ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-aryl-heterocycloalkyl-aryl, and substituted or unsubstituted heteroaryl-aryl-heterocycloalkyl-aryl. In certain embodiments, each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl. In specific embodiments, any of the groups described herein as being substituted are substituted with 1-5 substituents. In more specific embodiments, the substituents are selected from hydrogen, halo, hydroxy, thioxy, amino, amides, acetyl, alkoxy, alkylthioxy, cyano, alkyl, hydroxyalkyl, aryl, heteroaryl, cycloalkyl, and heterocycloalkyl.

In specific embodiments, the compound of Formula II is selected from sulconazole, econazole, clotrimazole, miconazole, bifonazole, fenticonizole, isoconazole, oxiconazole, sertaconazole, tioconazole, fluconazole, butoconazole, isavuconazole, ravuconazole, voriconazole, albaconazole, terconazole, posaconazole, and pharmaceutically acceptable salts thereof. In a more specific embodiment, the pharmaceutically acceptable salt thereof is a nitrate thereof.

In some embodiments, the neuroprotectant described herein is a compound of Formula III, or a pharmaceutically acceptable salt, steroisomer, tautomer, metabolite or solvate thereof:

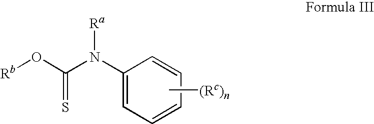

In some embodiments, the term R^{a} is selected from hydrogen and alkyl. In certain embodiments, R^{b} is selected from substituted or unsubstituted phenyl, substituted and unsubstituted naphthyl, substituted and unsubstituted 5,6,7,8-tetrahydronaphthyl, wherein m is 1, 2 or 3. In specific embodiments, the naphthyl is naphth-2-yl. In other specific embodiments, the 5,6,7,8-tetrahydronaphthyl is 5,6,7,8-tetrahydronaphth-2-yl. In certain embodiments, the 5,6,7,8-tetrahydronaphthyl group is a bridged 5,6,7,8-tetrahydronaphthyl group, e.g.,

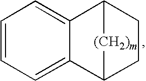

wherein m is 1, 2 or 3. In certain embodiments, each R^{c} is independently selected from hydrogen, halo, nitro, cyano, haloalkyl, perhaloalkyl, substituted and unsubstituted aryl, substituted and unsubstituted alkyl, substituted and unsubstituted alkoxy, —COR^{d}, and —NCOR^{d}. In some embodiments, each R^{d} is independently selected from substituted and unsubstituted alkyl, substituted and unsubstituted cycloalkyl, substituted and unsubstituted heterocycloalkyl, and substituted and unsubstituted aryl. In specific embodiments, any of the groups described herein as being substituted are substituted with 1-5 substituents. In more specific embodiments, the substituents are selected from hydrogen, halo, hydroxy, amino, acetyl, alkoxy, alkyl, aryl, heteroaryl, cycloalkyl, and heterocycloalkyl.

In certain embodiments, any of the neuroprotectants and/or antifungal compounds disclosed herein are utilized in a method of treating a neurodegenerative disorder, peripheral neuropathy or neuropathic pain. In certain embodiments, the method comprises administering a therapeutically effective amount of the compound to an individual in need thereof. In some embodiments, provided herein is a method of reducing neuronal or glial cell death by contacting a plurality of neurons or glial cells in need of protection from cell death with an effective amount of any neuroprotectant and/or antifungal compound disclosed herein.

The neuroprotective compounds described herein can be identified or characterized in an in vitro cellular assay, e.g., an assay that determines the viability of neurons or glia in the presence of a cytotoxic challenge and one or more concentrations of a candidate neuroprotective compound. Such assays are useful for identifying and testing the in vitro neuroprotective potency of candidate neuroprotective compounds.

For example, a cell viability assay can be utilized to detect and/or measure the ability of a compound disclosed herein to protect neuronal and/or glial cells. In some embodiments, mixed hippocampal cell cultures are prepared as described in, e.g., Haughey et al. (1999), J Neurochem, 73(4):1363-1374, and Haughey et al (2004), J Neurosci, 24(1):257-268. Subsequently, the cultures are pretreated for one hour in the presence of a candidate neuroprotective agent and then incubated with a cytotoxic agent for about 18 hours. Examples of cytotoxic agents include, but are not limited to, oxidative stressors (e.g., 3-nitropropionic acid (3-NP) or H₂O₂),.excitatory amino acids (e.g., kainate), or neurotoxic proteins (e.g., Hiv Tat, or Aβ peptides). If the survival of cell cultures in the presence of the candidate compound and the cytotoxic agent (e.g., 3-NP) is significantly greater than that of cultures incubated with the cytotoxic agent alone, the candidate test compound is considered to have neuroprotective activity. The level of protection (“percent protection”) provided by the test compound at a given test concentration in vitro can be expressed as:

$$\frac{[ \begin{matrix}( Cell Viability with Cytotoxic agent + Test Compound ) - \\ ( Cell Viability with Cytotoxic agent alone )\end{matrix} ]}{[ \begin{matrix}( Control Cell Viability ( medium alone ) - \\ ( Cell Viability with Cytotoxic agent alone )\end{matrix} ]} \times 100$$

In some embodiments, at a concentration of 10 μM, a neuroprotective compound described herein, provides at least 6% protection against a cytotoxic agent, including, e.g., at least 5%, 7%, 9%, 10%, 12%, 13%, 15%, 18%, 19%, 20%, 22%, 23%, 26%, 28%, 32%, 33%, 36%, 38%, 39%, 42%, 45%, 46%, 50%, 54%, 56%, 58%, 61%, 65%, 66%, 69%, 70%, 77%, 82%, 84%, 97%, or any other percent from at least 6% to 100% protection against the cytotoxic agent.

In some embodiments, at a concentration of 10 μM, a neuroprotective compound described herein, provides greater than 100% protection, which signifies that, in addition to blocking cytotoxic agent-induced cell death, the neuroprotective compound also inhibits spontanteous cell death in the control cell cultures (i.e., cultures exposed to medium alone). In some embodiments, a neuroprotective compound described herein provides, e.g., greater than 110%, greater than 120%, greater than 130%, greater than 140%, or greater than 150% protection.

Any suitable methods for determining cell viability may be used. In some embodiments, cell viability is assessed based on the “MTT” assay method described in Mosmann (1983), J Immunol Methods, 65(1-2):55-63, or a variant thereof. This assay is based on the ability of a mitochondrial dehydrogenase enzyme from viable cells to cleave the tetrazolium rings of the pale yellow MTT and form dark blue formazan crystals, which are trapped in cells and readily quantified by ELISA spectrophotometry.

High throughput cell viability assays may be used, and are particularly useful for detecting or measuring, with routine effort, the ability of the neuroprotective compounds described herein to protect neuronal or glial cells. See, e.g., J Biomol Screen, 9(6):506-515; Carrier et al. (2006), J Neurosci Methods, 154(1-2):239-244; See, e.g., In addition, high throughput systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay.

The neuroprotective compounds described herein, can be used for the manufacture of a medicament for treating any of the foregoing neurodegenerative conditions (e.g., multiple sclerosis, Alzheimer's disease, stroke, or spinal cord injury).

In some embodiments, a neuroprotective compound used for the methods described herein in vitro ED₅₀ for neuroprotection of less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM).

### Examples of Pharmaceutical Compositions and Methods of Administration

In certain embodiments, provided herein are pharmaceutical solid dosage forms. In some embodiments, pharmaceutical solid dosage forms described herein include any of the neuroprotective compounds described herein, and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of any of the neuroprotective compounds described herein. In one embodiment, some or all of the particles of any of the neuroprotective compounds described herein are coated. In another embodiment, some or all of the particles of any of the neuroprotective compounds described herein are microencapsulated. In still another embodiment, the particles of any of the neuroprotective compounds described herein are not microencapsulated and are uncoated.

Pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., “The Theory and Practice of Industrial Pharmacy” (1986). Other methods include, e.g., spray drying, pan coating, melt granulation, granulation, fluidized bed spray drying or coating (e.g., wurster coating), tangential coating, top spraying, tableting, extruding and the like.

Provided herein are pharmaceutical compositions that include one or more neuroprotective compounds described herein and a pharmaceutically acceptable diluent(s), excipient(s), or carrier(s). In addition, in some embodiments, the compounds described herein are administered as pharmaceutical compositions in which compounds described herein are mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In additional embodiments, the pharmaceutical compositions contain other therapeutically valuable substances.

In certain embodiments, compositions also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

A pharmaceutical composition, as used herein, refers to a mixture of any of the neuroprotective compounds described herein, such as, for example, an antifungal agent or a compound of Formula I, Formula II, or Formula III with other chemical components, such as carriers, stabilizers, diluents, dispersing agents, suspending agents, thickening agents, and/or excipients. In certain instances, the pharmaceutical composition facilitates administration of the compound to an organism. In certain embodiments, practicing the methods of treatment or use provided herein, therapeutically effective amounts of compounds described herein are administered, e.g., in a pharmaceutical composition to a mammal having a neurodegenerative condition, disease, or disorder to be treated. In certain embodiments, the mammal is a human. In certain instances, a therapeutically effective amount varies widely depending on the severity and stage of the condition, the age and relative health of the subject, the potency of the compound used and other factors. In various embodiments, the compounds are used singly or in combination with one or more therapeutic agents as components of mixtures.

In various embodiments, the pharmaceutical formulations described herein are administered to a subject by any one or more of multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In certain embodiments, pharmaceutical compositions including any of the neuroprotective compounds described herein is manufactured by any acceptable means including, by way of example only, by means of mixing, dissolving, granulating, dragee-making, levigating, emulsifying, encapsulating, entrapping or compression processes.

In some embodiments, the pharmaceutical compositions will include at least one of the neuroprotective compounds described herein described herein, such as, for example, an antifungal agent or a compound of Formula I, Formula II, or Formula III, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Bioavailability” refers to the percentage of the weight of compounds disclosed herein, such as, antifungal agents or compounds of Formula I, Formula II, or Formula III, that is delivered into the general circulation of the animal or human being studied. The total exposure (AUC(0-∞)) of a drug when administered intravenously is usually defined as 100% bioavailable (F %). “Oral bioavailability” refers to the extent to which compounds disclosed herein, such as, antifungal agents or compounds of Formula I, Formula II, or Formula III that are absorbed into the general circulation when the pharmaceutical composition is taken orally as compared to intravenous injection.

“Blood plasma concentration” refers to the concentration of compounds disclosed herein, such as, antifungal agents or compounds of Formula I, Formula II, or Formula III, in the plasma component of blood of a subject. It is understood that the plasma concentration of compounds disclosed herein may vary significantly between subjects, due to variability with respect to metabolism and/or possible interactions with other therapeutic agents. In accordance with one embodiment disclosed herein, the blood plasma concentration of the compounds disclosed herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound disclosed herein may vary from subject to subject.

“Carrier materials” include any commonly used excipients in pharmaceutics and should be selected on the basis of compatibility with compounds disclosed herein, such as, antifungal agents or compounds of Formula I, Formula II, or Formula III, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. In certain embodiments, plasticizers such as cellulose or triethyl cellulose are used as dispersing agents. Dispersing agents particularly useful in liposomal dispersions and self-emulsifying dispersions include dimyristoyl phosphatidyl choline, natural phosphatidyl choline from eggs, natural phosphatidyl glycerol from eggs, cholesterol and isopropyl myristatc.

The term “diluent” refers to chemical compounds that are used to dilute the compound of interest prior to delivery. In certain instances, diluents are used to stabilize compounds because they provide a more stable environment. In some embodiments, salts dissolved in buffered solutions (which also can provide pH control or maintenance) are utilized as diluents, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium Phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. In some embodiments, “Disintegration agents or disintegrants” are utilized, e.g., to facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH 101, Avicel® PH102, Avicel® PH105, Elcema® P100, Emcocel®, Vivacel®, Ming Tia®, and Solka-Floc®, methylcellulose carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

The term “therapeutically effective amount” or “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. In certain embodiments, an appropriate “effective” amount of a compound described herein in any individual is determined using techniques, such as a dose escalation study.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or 1 of blood serum, absorbed into the bloodstream after administration. As used herein, measurable plasma concentrations are typically measured in ng/ml or μg/ml.

The terms “neuroprotective,” “neuroprotection,” or “neuroprotectant,” as used herein refer to the ability of an agent (e.g., a compound described herein) to significantly prevent or reduce the occurrence of spontaneous or induced death (e.g., by apoptosis) of neurons or glia in vitro or in vivo, relative to the ability of a control reagent (e.g., cell culture medium, or a drug vehicle such as DMSO).

A “prognostic biomarker,” as referred to herein, is any molecular, biochemical, metabolic, cellular, or structural entity (or a ratio of such entities), the presence or level of which relates to a likelihood of developing, suffering from, or having a relapse of a pathological condition.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

The term “subject” or “individual” as used herein, refers to an animal which is the object of treatment, observation or experiment. By way of example only, a subject may be, but is not limited to, a mammal including, but not limited to, a human.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Treating” includes treating, preventing, delaying the onset of, reducing the symptoms of, reducing the risk of, maintaining or increasing the length of remission of, or delaying, inhibiting or slowing the progression of a disorder, including a neurodegenerative disorder, peripheral neuropathy or neuropathic pain.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In certain embodiments, the compositions described herein are formulated for administration to a subject via any means including, but not limited to, oral, parenteral (e.g., intravenous, subcutaneous, or intramuscular), buccal, intranasal, rectal or transdermal administration routes. As used herein, the term “subject” or “individual” is used to mean an animal, preferably a mammal, including a human or non-human. The terms patient, individual and subject are used interchangeably herein.

Moreover, in some embodiments, the pharmaceutical compositions described herein, which include any of the compounds described herein, e.g., an antifungal agent or compound of Formula I, Formula II, or Formula III, is formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross linked croscarmellose sodium, polyvinylpyrrolidone, agar, or alginic acid or a salt thereof such as sodium alginate.

In some embodiments, dragee cores are provided with suitable coatings. For this purpose, in some embodiments, concentrated sugar solutions are used, which optionally contain gum arabic, talc, polyvinylpyrrolidone, carbopol gel, polyethylene glycol, and/or titanium dioxide, lacquer solutions, and suitable organic solvents or solvent mixtures. In certain instances, dyestuffs or pigments are added to the tablets or dragee coatings for identification or to characterize different combinations of active compound doses.

In certain embodiments, pharmaceutical preparations which are used orally include push fit capsules made of gelatin, as well as soft, sealed capsules made of gelatin and a plasticizer, such as glycerol or sorbitol. In some embodiments, the push fit capsules contain the active ingredients in admixture with filler such as lactose, binders such as starches, and/or lubricants such as talc or magnesium stearate and, optionally, stabilizers. In soft capsules, the active compounds are optionally dissolved or suspended in suitable liquids, such as fatty oils, liquid paraffin, or liquid polyethylene glycols. In addition, stabilizers are optionally added. All formulations for oral administration should be in dosages suitable for such administration.

In some embodiments, the solid dosage forms disclosed herein is in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of any compound described herein, e.g., an antifungal agent or a compound of Formula I, Formula II, or Formula III, with one or more pharmaceutical excipients to form a bulk blend composition. When referring to these bulk blend compositions as homogeneous, it is meant that the particles of the compound are dispersed evenly throughout the composition so that the composition may be readily subdivided into equally effective unit dosage forms, such as tablets, pills, and capsules. In some embodiments, the individual unit dosages also include film coatings, which disintegrate upon oral ingestion or upon contact with diluent. In various embodiments, these formulations are manufactured by any acceptable pharmacological technique.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with the neuroprotective compounds disclosed herein, which sufficiently isolate the compound from other non-compatible excipients. Materials compatible with the neuroprotective compounds described herein includes those that delay the release of the compound in vivo.

Exemplary microencapsulation materials useful for delaying the release of the formulations including a neuroprotective compound described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

In certain embodiments, microencapsulated compounds described herein are formulated. Such methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In additional embodiments, several chemical techniques, e.g., complex coacervation, solvent evaporation, polymer-polymer incompatibility, interfacial polymerization in liquid media, in situ polymerization, in-liquid drying, and desolvation in liquid media are optionally used. Furthermore, other methods such as roller compaction, extrusion/spheronization, coacervation, or nanoparticle coating are optionally used.

In one embodiment, the particles of neuroprotective compounds disclosed herein are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds disclosed herein are plasticized (coated) with one or more layers. Illustratively, a plasticizer is generally a high boiling point solid or liquid. In some embodiments, suitable plasticizers are added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In some embodiments, the pharmaceutical solid oral dosage forms including formulations described herein, which include a neuroprotective compound described herein, are formulated to provide a controlled release of the compound. Controlled release refers to the release of the compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. In certain instances, such release rates provide therapeutically effective levels of agent for an extended period of time and thereby provide a longer period of pharmacologic response while minimizing side effects as compared to conventional rapid release dosage forms. In some instances, such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine of the gastrointestinal tract. In certain embodiments, the enteric coated dosage form is a compressed or molded or extruded tablet/mold (coated or uncoated) containing granules, powder, pellets, beads or particles of the active ingredient and/or other composition components, which are themselves coated or uncoated. In some embodiments, the enteric coated oral dosage form is a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. In certain embodiments coatings are applied to a sufficient thickness such that the entire coating does not dissolve in the gastrointestinal fluids at pH below about 5, but does dissolve at pH about 5 and above. In some embodiments, any anionic polymer exhibiting a pH-dependent solubility profile is used as an enteric coating in the practice of the present invention to achieve delivery to the lower gastrointestinal tract. In some embodiments the polymers for use in the present invention are anionic carboxylic polymers.

In other embodiments, the formulations described herein, which include any of the compounds described herein, e.g., an antifungal agent or compound of Formula I, Formula II, or Formula III, are delivered using a pulsatile dosage form. A pulsatile dosage form is capable of providing one or more immediate release pulses at predetermined time points after a controlled lag time or at specific sites. In some embodiments, pulsatile dosage forms including the formulations described herein, which include any of the compounds described herein, e.g., an antifungal agent or compound of Formula I, Formula II, or Formula III, are administered using a variety of pulsatile formulations. For example, such formulations include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284. In one embodiment, the controlled release dosage form is pulsatile release solid oral dosage form including at least two groups of particles, (i.e. multiparticulate) each containing the formulation described herein. The first group of particles provides a substantially immediate dose of any of the compounds described herein, e.g., an antifungal agent or compound of Formula I, Formula II, or Formula III, upon ingestion by a mammal. In some embodiments, the first group of particles is either uncoated or include a coating and/or sealant and the second group of particles includes coated particles, which includes from about 2% to about 75%, preferably from about 2.5% to about 70%, and more preferably from about 40% to about 70%, by weight of the total dose of any of the compounds described herein, e.g., an antifungal agent or compound of Formula I, Formula II, or Formula III, in said formulation, in admixture with one or more binders. In certain embodiments, the coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes any of the compounds described herein, e.g., an antifungal agent or compound of Formula I, Formula II, or Formula III.

Additional examples of controlled release delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, liquid formulation dosage forms for oral administration are aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of any of the compounds described herein, e.g., an antifungal agent or compound of Formula I, Formula II, or Formula III, the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystalline inhibitor.

In certain embodiments, the aqueous suspensions and dispersions described herein remain in a homogenous state, as defined in The USP Pharmacists’ Pharmacopeia (2005 edition, chapter 905), for at least 4 hours. In certain embodiments, the homogeneity is determined by a sampling method consistent with regard to determining homogeneity of the entire composition. In one embodiment, an aqueous suspension is re-suspended into a homogenous suspension by physical agitation lasting less than 1 minute. In another embodiment, an aqueous suspension is re-suspended into a homogenous suspension by physical agitation lasting less than 45 seconds. In yet another embodiment, an aqueous suspension is re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybcnzoic acid such as butylparaben, alcohols such as ethyl alcohol or benzyl alcohol, phenolic compounds such as phenol, or quaternary compounds such as benzalkonium chloride. In certain embodiments, preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. In certain instances, the concentration of the viscosity enhancing agent depends upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion comprises a sweetening agent and/or flavoring agent in a concentration ranging from about 0.001% to about 1.0% the volume of the aqueous dispersion. In another embodiment, the aqueous liquid dispersion comprises a sweetening agent and/or flavoring agent in a concentration ranging from about 0.005% to about 0.5% the volume of the aqueous dispersion. In yet another embodiment, the aqueous liquid dispersion comprises a sweetening agent or flavoring agent in a concentration ranging from about 0.01% to about 1.0% the volume of the aqueous dispersion.

In addition to the additives listed above, the liquid formulations also includes, in various embodiments, inert diluents such as water or other solvents, solubilizing agents, and/or emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein are self-emulsifying drug delivery systems (SEDDS). Emulsions are dispersions of one immiscible phase in another, usually in the form of droplets. In certain instances, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. In certain embodiments, SEDDS provide improvements in the bioavailability of hydrophobic active ingredients. Methods of producing self-emulsifying dosage forms include, but are not limited to, for example, U.S. Pat. Nos. 5,858,401, 6,667,048, and 6,960,563.

There is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein.

In some embodiments, intranasal formulations of any of the compounds described herein, e.g., an antifungal agent or compound of Formula I, Formula II, or Formula III, are prepared by adapting the methods described in U.S. Pat. Nos. 4,476,116, 5,116,817 and 6,391,452. Formulations that include any of the compounds described herein, e.g., an antifungal agent or compound of Formula I, Formula II, or Formula III, which are prepared according to these and other techniques are prepared as solutions in saline, employing benzyl alcohol or other suitable preservatives, fluorocarbons, and/or other solubilizing or dispersing agents. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients can be found in REMINGTON: THE SCIENCE AND PRACTICE OF PHARMACY, 21st edition, 2005, a standard reference in the field. The choice of suitable caniers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. In some embodiments, the nasal dosage form is isotonic with nasal secretions.

In certain embodiments, any of the compounds described herein, e.g., an antifungal agent or compound of Formula I, Formula II, or Formula III is administration by inhalation, e.g., as an aerosol, a mist or a powder. In certain embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is optionally determined by providing a valve to deliver a metered amount. In some embodiments, capsules and cartridges of, such as, by way of example only, gelatin for use in an inhaler or insufflator are formulated containing a powder mix of the compound described herein and a suitable powder base such as lactose or starch.

In some embodiments, buccal formulations that include any of the compounds described herein, e.g., an antifungal agent or compound of Formula I, Formula II, or Formula III, are administered using a variety of formulations. For example, such formulations include, but are not limited to, U.S. Pat. Nos. 4,229,447, 4,596,795, 4,755,386, and 5,739,136. In addition, the buccal dosage forms described herein optionally includes a bioerodible (hydrolysable) polymeric carrier that also serves to adhere the dosage form to the buccal mucosa. In certain instances, the buccal dosage form is fabricated so as to erode gradually over a predetermined time period, wherein the delivery of any of the compounds described herein, e.g., an antifungal agent or compound of Formula I, Formula II, or Formula III, is provided essentially throughout. In some instances, buccal drug delivery avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with any of the compounds described herein, e.g., an antifungal agent or compound of Formula I, Formula II, or Formula III, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and copolymers, e.g., those known as “carbomers” (Carbopol®, which may be obtained from B.F. Goodrich, is one such polymer). Other components are optionally incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels.

In certain embodiments, transdermal formulations described herein are administered using a variety of devices that include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

In some embodiments, the transdermal dosage forms described herein incorporate certain pharmaceutically acceptable excipients. In one embodiments, the transdermal formulations described herein include at least three components: (1) a formulation of any of the compounds described herein, e.g., an antifungal agent or compound of Formula I, Formula II, or Formula III; (2) a penetration enhancer; and (3) an aqueous adjuvant. In addition, transdermal formulations optionally include additional components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In certain embodiments, such patches are constructed for continuous, pulsatile, or on demand delivery of pharmaceutical agents. In further embodiments, transdermal delivery of the compounds described herein are accomplished by means of iontophoretic patches and the like. In certain instances, transdermal patches provide controlled delivery of any of the compounds described herein, e.g., an antifungal agent or compound of Formula I, Formula II, or Formula III. The rate of absorption can be slowed by using rate-controlling membranes or by trapping the compound within a polymer matrix or gel. Conversely, absorption enhancers are optionally used to increase absorption. An absorption enhancer or carrier includes absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In certain embodiments, formulations that include any of the compounds described herein, e.g., an antifungal agent or compound of Formula I, Formula II, or Formula III, suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection optionally contain additives such as preserving, wetting, emulsifying, and dispensing agents. In certain embodiments, prevention of the growth of microorganisms is ensured by various antibacterial and antifungal agents, such as parabens, chlorobutanol, phenol, sorbic acid, and the like. In some embodiments, it is desirable to include isotonic agents, such as sugars, sodium chloride, and the like. In certain embodiments, prolonged absorption of the injectable pharmaceutical form is acheived through the use of agents delaying absorption, such as aluminum monostearate and gelatin.

In some embodiments, any of the compounds described herein, e.g., an antifungal agent or compound of Formula I, Formula II, or Formula III, are formulated for intravenous injection. In such formulations, any of the compounds described herein, e.g., an antifungal agent or compound of Formula I, Formula II, or Formula III, are formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations optionally include aqueous or nonaqueous solutions, preferably with physiologically compatible buffers or excipients.

Parenteral injections include bolus injection and continuous infusion. In some embodiments, formulations for injection are presented in unit dosage form, e.g., in ampoules or in multi-dose containers, with an added preservative. In certain embodiments, the pharmaceutical composition described herein are in a form suitable for parenteral injection as a sterile suspensions, solutions or emulsions in oily or aqueous vehicles, and optionally contain formulatory agents such as suspending, stabilizing and/or dispersing agents. Pharmaceutical formulations for parenteral administration include aqueous solutions of the active compounds in water-soluble form. Additionally, suspensions of the active compounds are prepared, in some embodiments, as appropriate oily injection suspensions. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, the suspension also contains suitable stabilizers or agents which increase the solubility of the compounds to allow for the preparation of highly concentrated solutions. In some embodiments, the active ingredient is in powder form for constitution with a suitable vehicle, e.g., sterile pyrogen-free water, before use.

In certain embodiments, delivery systems for pharmaceutical compounds are employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein is administered topically and is formulated into one or more of a variety of topically administrable compositions, such as solutions, suspensions, lotions, gels, pastes, medicated sticks, balms, creams or ointments. In certain embodiments, such pharmaceutical compounds contain solubilizers, stabilizers, tonicity enhancing agents, buffers and/or preservatives.

Topical pharmaceutical formulations described herein comprise a pharmaceutically acceptable topical carrier, and an antifungal compound described herein. The topical pharmaceutical formulation is in any form suitable for application to the skin and optionally comprises, for example, a cream, lotion, solution, gel, ointment, paste, plaster, paint, bioadhesive, or the like, and/or includes liposomes, micelles, and/or microspheres.

Ointments are semisolid preparations that are typically based on petrolatum or other petroleum derivatives. The specific ointment base to be used is one that provides for drug delivery, and, optionally provides for other desired characteristics as well, e.g., emolliency or the like. As with other carriers or vehicles, an ointment base should be inert, stable, nonirritating and nonsensitizing. Ointment bases are typically grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Water-soluble ointment bases are optionally prepared from polyethylene glycols of varying molecular weight.

Creams are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Gels are semisolid, suspension-type systems. Single-phase gels contain organic macromolecules distributed substantially uniformly throughout the carrier liquid, which is typically aqueous, but also, optionally, contains an alcohol and, optionally, an oil. Optional “organic macromolecules,” i.e., gelling agents, include crosslinked acrylic acid polymers such as the “carbomer” family of polymers, e.g., carboxypolyalkylenes that may be obtained commercially under the Carbopol® trademark. Further examples are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin are optionally added, or the gelling agent are dispersed by trituration, mechanical mixing or stirring, or combinations thereof.

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. In certain embodiments, lotions are used for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Plasters are comprised of a pasty mixture that is spread on the body, either directly or after being saturated into a base material such as cloth. Medications, including the bases of the invention, are dissolved or dispersed within the plaster to make a medicated plaster.

Topical formulations optionally contain a pharmaceutically acceptable viscosity enhancer and/or film former. A viscosity enhancer increases the viscosity of the formulation so as to inhibit its spread beyond the site of application.

Formulations are also optionally prepared with liposomes, micelles, and microspheres. Liposomes are microscopic vesicles having a lipid wall comprising a lipid bilayer. Generally, liposome formulations are preferred for poorly soluble or insoluble pharmaceutical agents. Liposomal preparations include cationic (positively charged), anionic (negatively charged) and neutral preparations. Cationic liposomes include N[1-2,3-dioleyloxy)propyl]-N,N,N-triethylammonium (DOTMA). Similarly, anionic and neutral liposomes are available, e.g., from Avanti Polar Lipids (Birmingham, Ala.), or are prepared using available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials are optionally mixed with DOTMA in appropriate ratios.

Micelles are comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10 and nonoxynol 30.

Microspheres are also optionally included in the topical pharmaceutical formulations described herein. Microspheres essentially encapsulate a drug or drug-containing formulation. Microspheres are generally, although not necessarily, formed from synthetic or naturally occurring biocompatible polymers, but may also include charged lipids such as phospholipids.

Various additives are optionally included in the topical pharmaceutical formulations described herein. For example, solvents, including relatively small amounts of alcohol, are optionally used to solubilize certain formulation components. The topical formulations also optionally include conventional additives such as opacifiers, antioxidants, fragrance, colorants, gelling agents, thickening agents, stabilizers, surfactants; antimicrobial agents, to prevent spoilage upon storage (e.g., methyl and propyl esters of p-hydroxybenzoic acid (i.e., methyl and propyl paraben), sodium benzoate, sorbic acid, imidurea, and combinations thereof).

The topical pharmaceutical formulations described herein optionally contain irritation-mitigating additives to minimize or eliminate the possibility of skin irritation or skin damage resulting from the composition. Suitable irritation-mitigating additives include, for example: -tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine.

In certain embodiments, the compounds described herein are formulated in rectal compositions such as enemas, rectal gels, rectal foams, rectal aerosols, suppositories, jelly suppositories, or retention enemas, containing suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In other embodiments, the formulations described herein, which include any of the compounds described herein, e.g., an antifungal agent or compound of Formula I, Formula II, or Formula III, are blood brain barrier-permeable (BBB-permeable) nanoparticle formulations. Methods of producing such BBB-permeable nanoparticle formulations include, but are not limited to, for example, U.S. Pat. Nos. 6,117,454 and 7,025,991.

### Combination Therapies

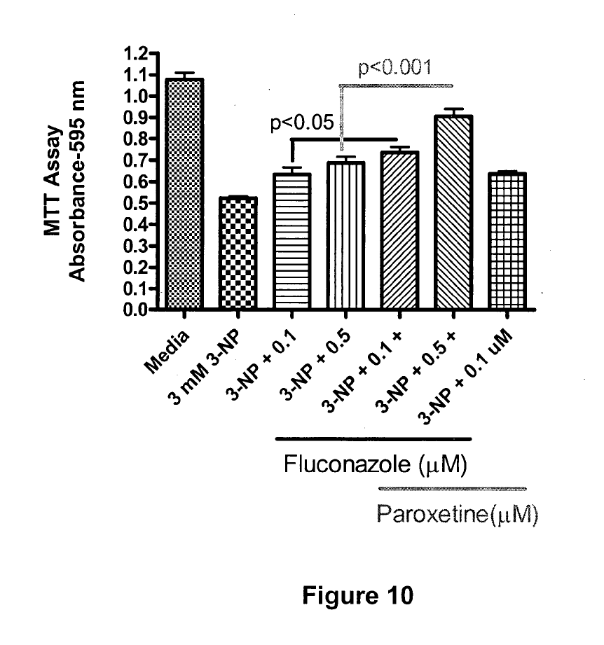

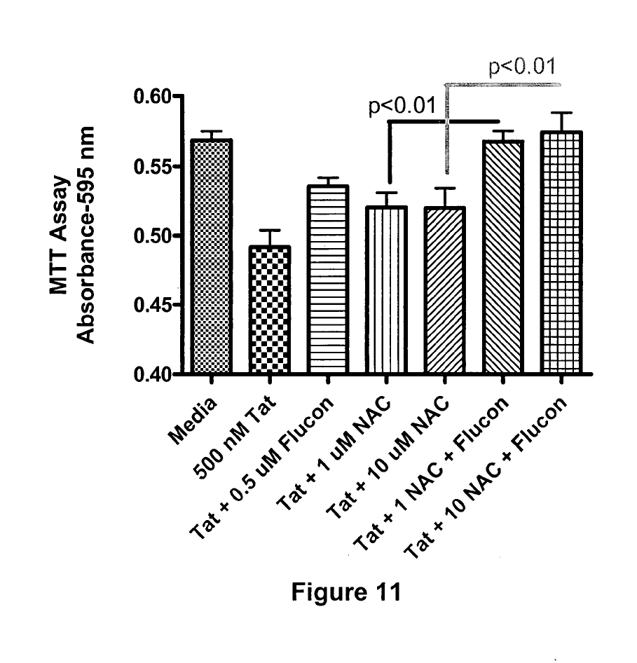

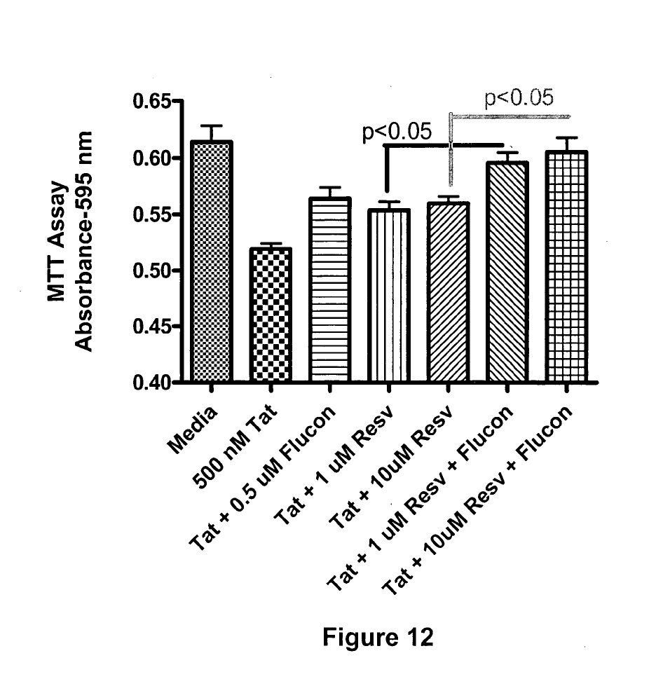

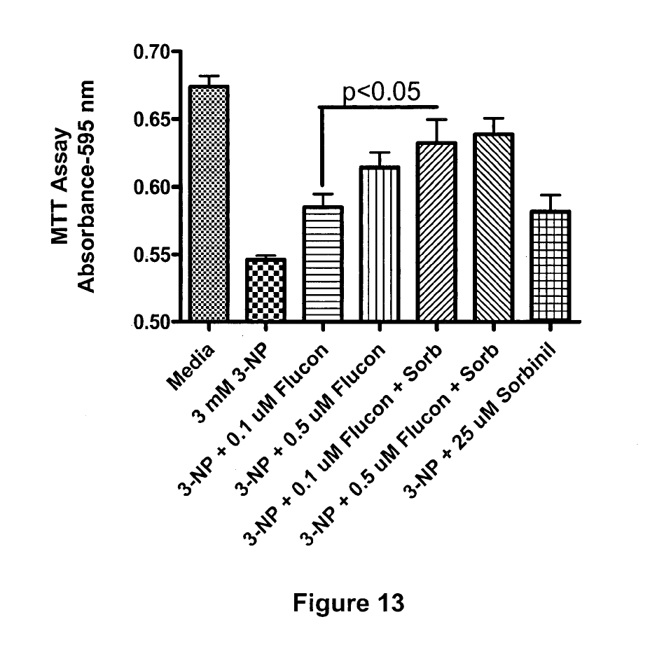

In certain embodiments, an antifungal agent, as described herein (including all formulae and specific examples), is combined with an additional agent. One example of an additional agent is an agent that operates via serotonergic mechanisms: an example of such a class of agents are the selective serotonin reuptake inhibitors (SSRIs). See FIG. 10 and figure legend. Another example of an additional agent is an agent that operates by blocking oxidative stress by neutralizing free radicals and reactive oxygen species: an example of such a class of agents are antioxidants. See FIGS. 11 and 12 and figure legends. Another example of an additional agent is an agent that operates by inducing neurotrophic factors such as NGF: an example of such a class of agents are aldose reductase inhibitors. See FIG. 13 and figure legend. An example of an SSRI is paroxetine; examples of antioxidants include N-acetyl cysteine and Resveratrol; an example of an aldose reductase inhibitor is Sorbinil. In certain embodiments, such combinations are administered simultaneously; in other embodiments, the antifungal agent is administered first, followed after a period of time by the second agent; in yet other embodiments, the antifungal agent is administered following the additional agent. Included herein are pharmaceutical compositions comprising an antifungal agent, as described herein, and an additional agent, as described herein. Also included herein are uses of antifungal agents, as described herein, and an additional agent, as described herein, for the combined neuroprotection or treatment of diseases or conditions associated with neurons or glia. Also included herein are methods of providing neuroprotection or the treatment of diseases or conditions associated with neurons or glia, comprising administering (either simultaneously or sequentially) an antifungal agent, as described herein, and an additional agent, as described herein, to a patient in need. In certain embodiments, the combination of the antifungal agent and the additional agent provides synergistic benefit to the patient, and in particular, synergistic neuroprotection and/or synergistic treatment of diseases or conditions associated with neurons or glia. In other embodiments, the combination of the antifungal agent and the additional agent provides a complementary benefit to the patient: by way of example only, an antioxidant alleviates or prevents oxidative stress by affecting ADP ribose mediated activation of TRPM2 channels.

### Examples of Methods of Dosing and Treatment Regimens

In certain embodiments, the compounds described herein are used in the preparation of medicaments for the treatment of neurodegenerative diseases or conditions that would benefit, at least in part, from neuroprotection. In addition, a method for treating any of the diseases or conditions described herein in a subject in need of such treatment, involves administration of pharmaceutical compositions containing at least one of the compounds described herein, e.g., an antifungal agent or compound of Formula I, Formula II, or Formula III, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In various embodiments, the compositions containing the compound(s) described herein are administered for prophylactic and/or therapeutic treatments, both of which are encompassed by a method of “treating”. In certain therapeutic applications, the compositions are administered to a patient already suffering from a disease or condition, in an amount sufficient to cure or at least partially arrest the symptoms of the disease or condition. In certain embodiments, amounts effective for this use depend on the severity and course of the disease or condition, previous therapy, the patient's health status, weight, and response to the drugs, and the judgment of the treating physician.

In certain prophylactic applications, compositions containing the compounds described herein are administered to a patient susceptible to or otherwise at risk of a particular disease, disorder or condition. In some embodiments, the precise prophylactically effective amount or dose depends on the patient's state of health, weight, and the like. In certain embodiments, when used in a patient, effective amounts for this use will depend on the severity and course of the disease, disorder or condition, previous therapy, the patient's health status and response to the drugs, and the judgment of the treating physician.

In certain instances wherein the patient's condition does not improve, upon the doctor's discretion the administration of the compounds is optionally administered chronically, that is, for an extended period of time, including throughout the duration of the patient's life in order to ameliorate or otherwise control or limit the symptoms of the patient's disease or condition.

In certain instances wherein the patient's status does improve, upon the doctor's discretion the administration of the compounds are given continuously; or alternatively, the dose of drug being administered is temporarily reduced or temporarily suspended for a certain length of time (i.e., a “drug holiday”). In various embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In certain embodiments, once improvement of the patient's conditions has occurred, a maintenance dose is administered if desired or if necessary. Subsequently, in some embodiments, the dosage or the frequency of administration, or both, is optionally reduced, as a function, e.g., of the symptoms, to a level at which the improved disease, disorder or condition is retained. In certain embodiments, however, patients may require intermittent treatment on a long-term basis upon any recurrence of symptoms.

In some embodiments, the amount of a given agent that correspond to such an amount varies depending upon factors such as the particular compound, disease or condition and its severity, the identity (e.g., weight) of the subject or host in need of treatment, but can nevertheless be determined according to the particular circumstances surrounding the case, including, e.g., the specific agent being administered, the route of administration, the condition being treated, and the subject or host being treated. In certain embodiments, doses employed for adult human treatment are in the range of about 0.01 to about 10 g per day, or about 1 mg to about 2 g per day, or about 200 mg to about 400 mg per day. In some embodiments, the desired dose is conveniently presented in a single dose or as divided doses administered simultaneously (or over a short period of time) or at appropriate intervals, for example as two, three, four or more sub-doses per day.

In certain embodiments, the pharmaceutical composition described herein is in unit dosage forms suitable for single administration of precise dosages. In certain unit dosage forms, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. In certain embodiments, aqueous suspension compositions are packaged in single-dose non-re-closeable containers. Alternatively, multiple-dose re-closeable containers are optionally used, in which case it is typical to include a preservative in the composition. In exemplary embodiments, formulations for parenteral injection are presented in a unit dosage form, which include, but are not limited to ampoules, or in multi-dose containers, with an added preservative.

The daily dosages appropriate for the compounds described herein described herein are from about 0.01 μg/kg to about 100 mg/kg, or about 1 mg/kg to about 10 mg/kg of body weight. The foregoing ranges are merely suggestive, as the number of variables in regard to an individual treatment regime is large, and considerable excursions from these recommended values are contemplated. In some embodiments, such dosages are altered depending on one or more of variables including, but not limited to, the activity of the compound used, the disease or condition to be treated, the mode of administration, the requirements of the individual subject, the severity of the disease or condition being treated, and the judgment of the practitioner.

In certain instances, toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

In some embodiments, the compounds and/or compositions described herein are administered on any suitable schedule including, by way of non-limiting example, three times a day, twice a day (b.i.d.), once a day, every other day, five times a week, four times a week, three times a week, twice a week or once a week.

### Combination Treatments

A neuroprotective compound compositions described herein is optionally used in combination with other therapeutic reagents that are selected for their therapeutic value for the condition to be treated. In general, the compositions described herein and, in embodiments where combinational therapy is employed, the two or more agents are either administered in a single composition or in separate and discrete compositions. Furthermore, because the two or more agents may have different physical and chemical characteristics, administered by different routes is also contemplated.

In certain instances, it is appropriate to administer at least one neuroprotective compound described herein in combination with another therapeutic agent. In certain embodiments, a neuroprotective compounds described herein has a side effect (e.g., of inducing nausea) in a patient. In some of such embodiments, it is appropriate to administer an agent for reducing or preventing the side effect (e.g., an anti-nausea agent) in combination with the initial therapeutic agent. In some embodiments, the therapeutic effectiveness of one of the compounds described herein is enhanced by administration of an adjuvant (i.e., by itself the adjuvant may have minimal therapeutic benefit, but in combination with another therapeutic agent, the overall therapeutic benefit to the patient is enhanced). In certain embodiments, the benefit experienced by a patient is increased by administering any the compounds described herein with one or more (e.g., one, two, or three) other therapeutic agents (which also includes a therapeutic regimen) that also have a therapeutic benefit. In any case, regardless of the disease, disorder or condition being treated, the overall benefit experienced by the patient includes additive and synergistic effects.

In various embodiments, the compounds are administered concurrently (e.g., simultaneously, essentially simultaneously or within the same treatment protocol) or sequentially, depending upon the nature of the disease, disorder, or condition, the condition of the patient, and the actual choice of compounds used.

In various embodiments, the multiple therapeutic agents (one of which is any of the compounds described herein, e.g., an antifungal agent or compound of Formula I, Formula II, or Formula III) are administered in any order, or even simultaneously. In certain embodiments, simultaneous administration includes administration of the multiple therapeutic agents in a single, unified form, and in multiple forms (by way of example only, either as a single pill or as two separate pills). In various instances, one of the therapeutic agents is given in multiple doses, or both may be given as multiple doses. If administration is not simultaneous, in certain embodiments, the timing between the multiple doses varies, by way of non-limiting example, from more than zero weeks to less than four weeks. In addition, the combination methods, compositions and formulations are not to be limited to the use of only two agents; the use of multiple therapeutic combinations is also envisioned.

In various embodiments, the pharmaceutical agents which make up the combination therapy disclosed herein are in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In certain instances, the two-step administration regimen may call for sequential administration of the active agents or spaced-apart administration of the separate active agents. In certain embodiments, the time period between the multiple administration steps ranges from, by way of non-limiting example, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In additional embodiments, the compounds described herein are used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. A compound is preferably administered as soon as is practicable after the onset of a disease or condition is detected or suspected, and for a length of time necessary for the treatment of the disease, such as, for example, from about 1 month to about 3 months. The length of treatment can vary for each subject. For example, the compound or a formulation containing the compound can be administered for at least 2 weeks, about 1 month to about 5 years, and from about 1 month to about 3 years. In certain embodiments, one or more of any of the neuroprotective compounds described herein is combined with at least one agent for treating multiple sclerosis, at least one agent for treating dementia (e.g., Alzheimer's Disease or AIDS-related Dementia), at least one agent for treating Parkinson's Disease, at least one agent for treating Amyotrophic Lateral Sclerosis, at least one agent for treating Huntington's Disease, at least one agent for treating an autoimmune inflammatory disorder, at least one agent for treating an allergic condition, at least one agent for treating a thromboembolic disorders, at least one agent for treating an HIV infection, at least one antipsychotic compound, at least one antiepileptic compound, at least one neuroprotective compound or composition (e.g., a neuroprotective compound that does not fall within the scope of the neuroprotective compounds disclosed herein), and combinations thereof.

Exemplary Therapeutic Agents for Use in Combination with a Neuroprotective Compound

### Agents for Treating Multiple Sclerosis

In certain embodiments, one or more neuroprotective compound is combined with or used together with one or more an agent for treating multiple sclerosis such as, by way of non-limiting example, Interferon β-1a, Interferon β-1b, glatiramer acetate (Copaxone®), mitoxantrone (Novantrone®), low dose naltrexone, Natalizumab (Tysabri®), Sativex®, Aimspro (Goats Serum), Trimesta (Oral Estriol), Laquinimod, FTY720 (Fingolimod), MBP8298, NeuroVax™, Tovaxin™, Revimmune, CHR-1103, BHT-3009, BG-12, Cladribine, daclizumab (Zenapax) Rituximab (Rituxan), cyclophosphamide, Campath, Fampridine-SR, MN-166, Temsirolimus, or RPI-78M. In certain instances, the combination is in a single composition or the combination is utilized in a combination treatment, wherein the combined agents are administered together or separately. In specific embodiments, such combinations are utilized in treating an individual that is suffering from or at risk of suffering from multiple sclerosis.

### Agents for Treating Dementia (e.g., Alzheimer's Disease or AIDS-Related Dementia)

In certain embodiments, one or more neuroprotective compound is combined with or used together with one or more an agent for treating dementia such as, by way of non-limiting example, Flurizan™ (MPC-7869, r flurbiprofen), memantine, galantamine, rivastigmine, donezipil, tacrine, Aβ₁₋₄₂ immunotherapy, resveratrol, (−)-epigallocatechin-3-gallate, statins, vitamin C, or vitamin E. In certain instances, the combination is in a single composition or the combination is utilized in a combination treatment, wherein the combined agents are administered together or separately. In specific embodiments, such combinations are utilized in treating an individual that is suffering from or at risk of suffering from dementia.

### Agents for Treating Parkinson's Disease

In certain embodiments, one or more neuroprotective compound is combined with or used together with one or more an agent for treating Parkinson's Disease such as, by way of non-limiting example, L-dopa, carbidopa, benserazide, tolcapone, entacapone, bromocriptine, pergolide, pramipexole, ropinirole, cabergoline, apomorphine, lisuride, selegiline, or rasagiline. In certain instances, the combination is in a single composition or the combination is utilized in a combination treatment, wherein the combined agents are administered together or separately. In specific embodiments, such combinations are utilized in treating an individual that is suffering from or at risk of suffering from Parkinson's Disease.

### Agents for Treating Amyotrophic Lateral Sclerosis

In certain embodiments, one or more neuroprotective compound is combined with or used together with one or more an agent for treating ALS such as, by way of non-limiting example, riluzole, insulin-like growth factor 1, or ketogenic diet. In certain instances, the combination is in a single composition or the combination is utilized in a combination treatment, wherein the combined agents are administered together or separately. In specific embodiments, such combinations are utilized in treating an individual that is suffering from or at risk of suffering from ALS.

### Agents for Treating Huntington's Disease

In certain embodiments, one or more neuroprotective compound is combined with or used together with one or more an agent for treating Huntington's Disease such as, by way of non-limiting example, dopamine receptor blockers, creatine, CoQ10, minocycline, exercise, antioxidants, antidepressants (notably, but not exclusively, selective serotonin reuptake inhibitors SSRIs, such as sertraline, fluoxetine, and paroxetine), dopamine antagonists, (e.g., tetrabenazine), or RNAi-mediated silencing of mutant Huntingtin expression. In certain instances, the combination is in a single composition or the combination is utilized in a combination treatment, wherein the combined agents are administered together or separately. In specific embodiments, such combinations are utilized in treating an individual that is suffering from or at risk of suffering from Huntington's Disease.

Agents for Treating Autoimmune, Inflammatory, or Allergic conditions

In certain embodiments, one or more neuroprotective compound is combined with or used together with one or more agent for treating autoimmune, inflammatory or allergic conditions such as, by way of non-limiting example, immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics. In certain instances, the combination is in a single composition or the combination is utilized in a combination treatment, wherein the combined agents are administered together or separately. In specific embodiments, such combinations are utilized in treating an individual that is suffering from or at risk of suffering from an autoimmune, inflammatory disease, or allergic condition that affects the nervous system.

### Agents for Treating Thromboembolic Disorders

In certain embodiments, one or more neuroprotective compound is combined with or used together with one or more agent for treating thromboembolic disorders such as, by way of non-limiting example, thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048. In certain instances, t combination is in a single composition or the combination is utilized in a combination treatment, wherein the combined agents are administered together or separately. In specific embodiments, such combinations are utilized in treating an individual that is suffering from or at risk of suffering from a thromboembolic disorder (e.g., stroke).

### Agents for Treating an HIV Infection

In certain embodiments, one or more neuroprotective compound is combined with or used together with one or more agent for treating an HIV infection such as, by way of non-limiting example, AZT (zidovudine, Retrovir), ddl (didanosine, Videx), 3TC (lamivudine, Epivir), d4T (stavudine, Zerit), abacavir (Ziagen), and FTC (emtricitabine, Emtriva), tenofovir (Viread), efavirenz (Sustiva), nevirapine (Viramune), lopinavir/ritonavir (Kaletra), indinavir (Crixivan), ritonavir (Norvir), nelfinavir (Viracept), saquinavir hard gel capsules (Invirase), atazanavir (Reyataz), amprenavir (Agenerase), fosamprenavir (Telzir), tipranavir (Aptivus), or T20 (enfuvirtide, Fuzeon). In certain instances, the combination is in a single composition or the combination is utilized in a combination treatment, wherein the combined agents are administered together or separately. In specific embodiments, such combinations are utilized in treating an individual that is suffering from or at risk of suffering from an HIV infection (e.g., suffering from AIDS).

### Antipsychotic Compounds

In certain embodiments, one or more neuroprotective compound is combined with or used together with one or more antipsychotic compound such as, by way of non-limiting example, clozapine, risperidone, olanzapine, quetiapine, ziprasidone, aripiprazole, paliperidone, sertindole, zotepine, amisulpride, bifeprunox, melperone, chlorpromazine (largactil, thorazine), fluphenazine, haloperidol, molindone, thiothixene, thioridazine, trifluoperazine, loxapine, perphenazine, prochlorperazine, pimozide, thiothixene, or zuclopenthixol. In certain instances, the combination is in a single composition or the combination is utilized in a combination treatment, wherein the combined agents are administered together or separately. In specific embodiments, such combinations are utilized in treating an individual that is suffering from or at risk of suffering from a psychotic disorder (e.g., schizophrenia).

### Antiepileptic Compounds

In certain embodiments, one or more neuroprotective compound is combined with or used together with one or more antiepileptic compound such as, by way of non-limiting example, carbamazepine, clobazam, clonazepam, ethosuximide, felbamate, fosphenytoin, flurazepam, gabapentin, lamotrigine, levetiracetam, oxcarbazepine, mephenytoin, phenobarbital, phenytoin, pregabalin, primidone, sodium valproate, tiagabine, topiramate, valproate semisodium, valproic acid, vigabatrin, diazepam, or lorazepam. In certain instances, the combination is in a single composition or the combination is utilized in a combination treatment, wherein the combined agents are administered together or separately. In specific embodiments, such combinations are utilized in treating an individual that is suffering from or at risk of suffering from epilepsy.

### Neuroprotective Compounds and Compositions

In certain embodiments, one or more neuroprotective compound is combined with or used together with one or more additional neuroprotective compound such as, by way of non-limiting example, resveratrol, GPI 1046, epigallocatechin gallate, α-lipoic acid, Omega-3 fatty acids (e.g., docosahexaenoic acid or eicosapentaenoic acid), Vitamin E (tocopherol), carnitine, cytidine diphosphocoline (citicholine), coenzyme Q10, curcumin, salviolonic acid B, folic acid, Gingko biloba extract, ginsenoside Rb1, ginsenoside Rg3, L-Glutathione, grape seed extract, lutein, zeaxanthin, methylcobalamin, N-acetyl-L-cysteine, pycnogenol, quercetin, or taurine.

### Examples

The following specific examples are to be construed as merely illustrative, and not ]imitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### Identification of Neuroprotective Compounds

We identify neuroprotective compounds using an in vitro neuroprotection assay as described in detail below. We screen a specific collection/library of compounds that we identified as potentially active agents. This particular collection contains 2000 compounds of which 50% are FDA-approved compounds, 30% are natural products, and 20% are other bioactive compounds. From these experiments, a number of agents (e.g., antifungal agents and/or agents having a chemical formula as set forth in Formula I, Formula II, or Formula III) were identified as neuroprotective agents.

### Example 2

### Pharmaceutical Compositions

### Example 2a

### Parenteral Composition

To prepare a parenteral pharmaceutical composition suitable for administration by injection, 100 mg of a water-soluble salt of a neuroprotective compound described herein (e.g., an antifungal agent or a compound of Formula I, Formula II, or Formula III), is dissolved in DMSO and then mixed with 10 mL of 0.9% sterile saline. The mixture is incorporated into a dosage unit form suitable for administration by injection.

### Example 2b

### Oral Composition

To prepare a pharmaceutical composition for oral delivery, 100 mg of a neuroprotective compound described herein (e.g., an antifungal agent or a compound of Formula I, Formula II, or Formula III), is mixed with 750 mg of starch. The mixture is incorporated into an oral dosage unit for, such as a hard gelatin capsule, which is suitable for oral administration.

### Example 2c

### Sublingual (Hard Lozenge) Composition

To prepare a pharmaceutical composition for buccal delivery, such as a hard lozenge, mix 100 mg of a neuroprotective compound described herein (e.g., an antifungal agent or a compound of Formula I, Formula II, or Formula III), with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 2d

### Inhalation Composition

To prepare a pharmaceutical composition for inhalation delivery, 20 mg of a neuroprotective compound described herein (e.g., an antifungal agent or a compound of Formula I, Formula II, or Formula III), is mixed with 50 mg of anhydrous citric acid and 100 mL of 0.9% sodium chloride solution. The mixture is incorporated into an inhalation delivery unit, such as a nebulizer, which is suitable for inhalation administration.

### Example 2e

### Rectal Gel Composition

To prepare a pharmaceutical composition for rectal delivery, 100 mg of a neuroprotective compound described herein (e.g., an antifungal agent or a compound of Formula I, Formula II, or Formula III), is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 2f

### Topical Gel Composition

To prepare a pharmaceutical topical gel composition, 100 mg of a neuroprotective compound described herein (e.g., an antifungal agent or a compound of Formula I, Formula II, or Formula III), is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topicl administration.

### Example 2g

### Ophthalmic Solution Composition

To prepare a pharmaceutical opthalmic solution composition, 100 mg of a neuroprotective compound described herein (e.g., an antifungal agent or a compound of Formula I, Formula II, or Formula III), is mixed with 0.9 g of NaCl in 100 mL of purified water and filterd using a 0.2 micron filter. The resulting isotonic solution is then incorporated into ophthalmic delivery units, such as eye drop containers, which are suitable for ophthalmic administration.

### Example 3

### Biological Assays and Analyses

### Example 3A

### Exemplary Modified Terpenoids are Neuroprotective against an Oxidative Stressor and a Neurotoxic Protein

We evaluate the protective efficacy of a number of antifungal agents against many different neurotoxins, ranging from the chemotoxic 6-OHDA, NMDA, 3-nitropropionic acid (3-NP), and viral proteins such as Tat and gp120. Thus, we provide an in vitro neuroprotection assay using rat mixed hippocampal cultures, in which we evaluate the protective efficacy of neuroprotective compounds described herein. The oxidative stressor 3-NP is used to elicit toxicity in the rat hippocampal cultures to mimic the oxidative damage, reactive oxygen species production and ensuing neurodegeneration resulting from HIV infection. Another measure of neurotoxicity which results from HIV infection is evaluated by exposure of the hippocampal cultures to HIV-1 Tat (Li et al (2005), Neurotox Res, 8(1-2):119-134).

Rat mixed hippocamal neuronal cultures are generated from freshly dissected rat hippocampi (embryonic day 18) in neurobasal media containing 5% fetal bovine serum and 2% B27 supplement. The cells are plated into 96 well plates at a density of 4×10⁵ cells/mL and routinely used on days 11-14 following culturing. Cell viability is assessed with MTT [3-(4,5-dimethylthiazol-2-yl)-2,5-diphenyltetrazolium bromide] assay. The MTT assay is based on the ability of a mitochondrial dehydrogenase enzyme from viable cells to cleave the tetrazolium rings of the pale yellow MTT and form dark blue formazan crystals. See Mosmann (1983), J Immunol Methods, 65(1-2):55-63. These crystals are largely impermeable to cell membranes, and thus accumulate within healthy cells. The resultant formazan precipitates are solubilized with DMSO and read on a multiwell scanning spectrophotometer (ELISA reader). The number of surviving cells is directly proportional to the level of the formazan product created.

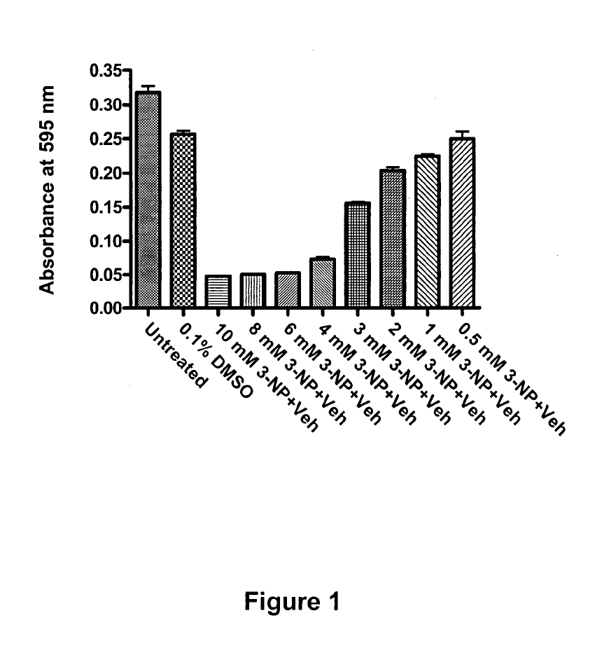

Mixed hippocampal cultures are incubated with 3-NP (0.5-10 mM) for 18 hours in a 0.1% DMSO vehicle and then assessed for viability using an MTT assay. As shown in FIG. 1, titration of 3-NP levels for neurotoxic effects demonstrated that 3 mM 3-NP treatment consistently induced 25-35% cytotoxicity in rat mixed hippocampal cultures compared to a 0.1% DMSO vehicle control.

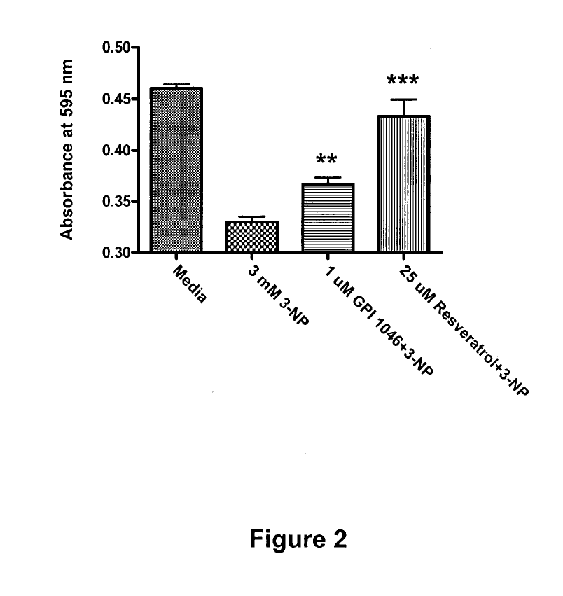

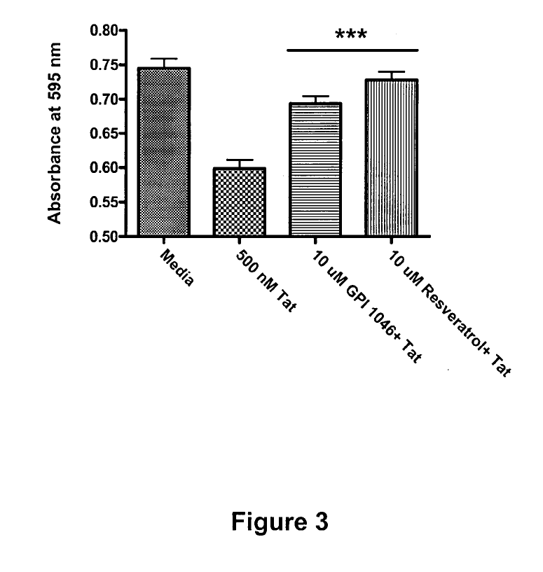

The assay system is validated using two neuroprotective agents, GPI 1046 and Resveratrol. Both of these compounds has demonstrated antioxidant and/or neuroprotective activities in numerous in vitro and in vivo assays (for review, see Poulter et al. (2004), Neuroscience, 128(1):1-6; Caporello, et al. (2006), J Neurochem, 98(1):146-155; Zamin et al. (2006), Neurobiol Dis, 24(1):176-182). Cultures are preincubated with GPI 1046 or Resveratrol for one hour prior to an 18 hour exposure to 3 mM 3-NP. These “positive control” neuroprotective compounds significantly protected rat neurons from oxidative damage elicited by 3-NP (FIG. 2) in the rat mixed hippocampal culture assay system described above. The same neuroprotective compounds are evaluated for efficacy against HIV-1 Tat protein toxicity using the same 1 hour preincubation protocol. As with the 3-NP neurotoxicity assay, these compounds protected hippocampal neurons from Tat toxicity as well (FIG. 3). These data indicated that the measurement of neuroprotection against 3-NP toxicity is a good indicator of protective activity against HIV-1 Tat toxicity.

Using the validated 3 mM 3-NP neurotoxicity assay described above, we evaluated the the neuroprotective efficacy of approximately 2000 compounds from the Spectrum Collection (MicroSource Discovery) as described in Example 1. Several of the neuroprotective compounds identified in this collection were, e.g., antifungal agents and/or compounds within the scope of Formula I, Formula II, or Formula III. As shown in Tables 2 and 3, a number of antifungal agents and/or compounds within the scope of Formula I, Formula II, or Formula III are identified as having neuroprotective activity against 3-NP. Neuroprotection vs 3NP is measured as a percentage of neural and/or glial cell that are protected from 3NP induced cell death after incubation with the neuroprotective agent (10 μM) for 60 minutes when compared to a 0.1% DMSO vehicle control.

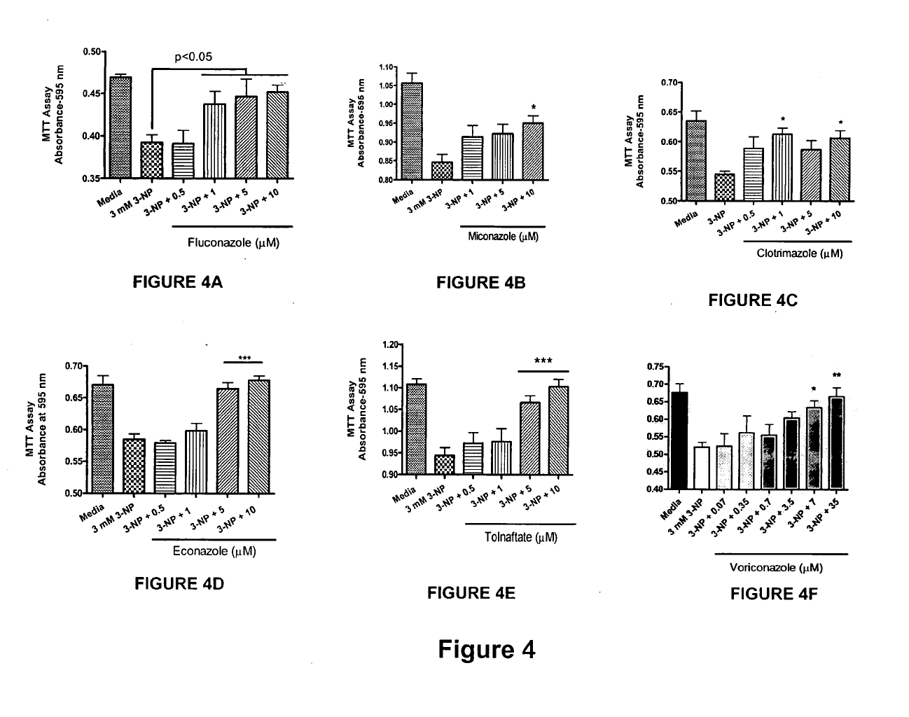

FIG. 4 illustrates 3-NP assay data for several of the neuroprotective compounds disclosed herein are provided. The neural and glial cells were preincubated with the neuroprotective compound for 1 hour followed by exposure to3 mM of the toxin for 18 hours. FIG. 4A illustrates the neuroprotective characteristic of fluconazole versus 3-NP at various concentrations (i.e., 0.5 μM, 1 μM, 5 μM, and 10 μM). FIG. 4B illustrates the neuroprotective characteristic of miconazole versus 3-NP at various concentrations (i.e., 1 μM, 5 μM, and 10 μM). FIG. 4C illustrates the neuroprotective characteristic of clotrimazole versus 3-NP at various concentrations (i.e., 0.5 μM, 1 μM, 5 μM and 10 μM). FIG. 4D illustrates the neuroprotective characteristic of econazole versus 3-NP at various concentrations (i.e., 0.5 μM, 1 μM, 5 μM, and 10 μM). FIG. 4E illustrates the neuroprotective characteristic of tolnaftate versus 3-NP at various concentrations (i.e., 0.5 μM, 1 μM, 5 μM, and 10 μM). FIG. 4F illustrates the neuroprotective characteristic of voriconazole versus 3-NP at various concentrations (i.e., 0.07 μM, 0.35 μM, 0.7 μM, 3.5 μM, 7 μM and 35 μM). Generally, the neuroprotective agents are observed protect the neural and/or glial cells in a concentration dependent manner and are observed to be active in the low to mid micromolar range against the oxidative stress mediated toxicity of 3-NP. Thus, the compounds disclosed herein protect neural and glial cells from oxidative stress.

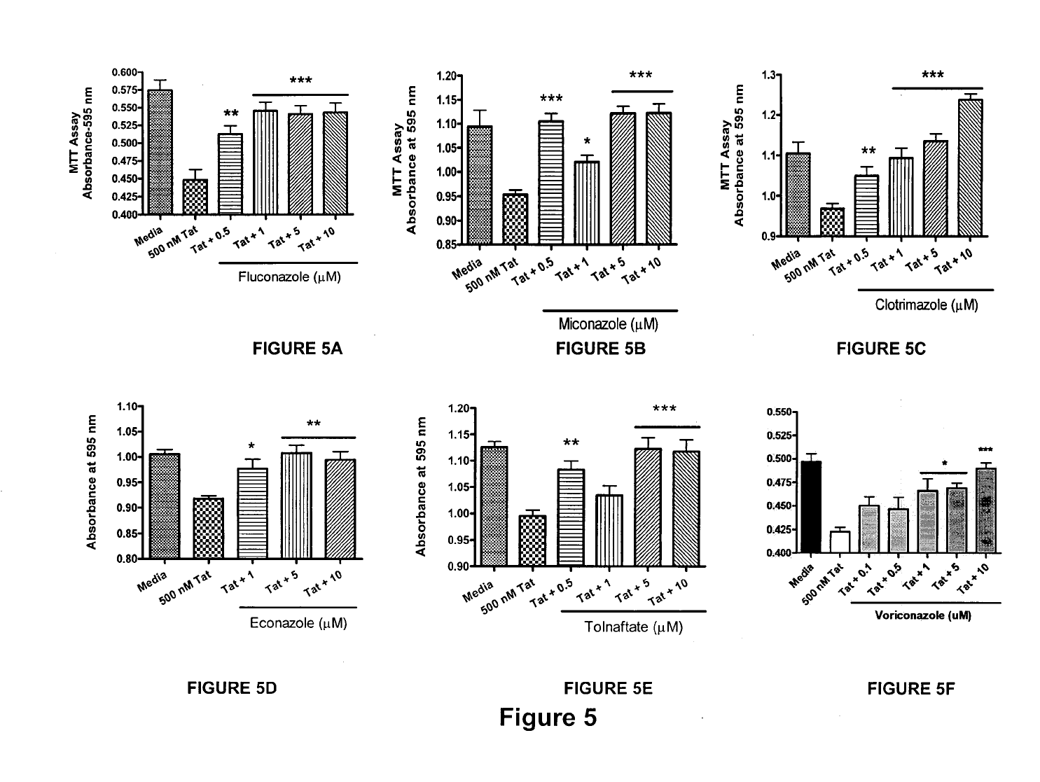

FIG. 5 illustrates the concentration dependent protection of hippocampal cultures from HIV-Tat toxicity with fluconazole, miconazole, clomitrazole, econazole, tolnaftate and voriconazole (FIGS. 5A-5F, respectively). The neural and glial cells were preincubated with the neuroprotective compound for 1 hour followed by exposure to 500 nM of Tat1-72 for 18 hours. Generally, the neuroprotective agents are observed to have neuroprotective character against HIV-Tat at concentration of about 500 nM. Thus, neuroprotective compounds disclosed herein also protect neural and/or glial cells from neurotoxic degeneration.

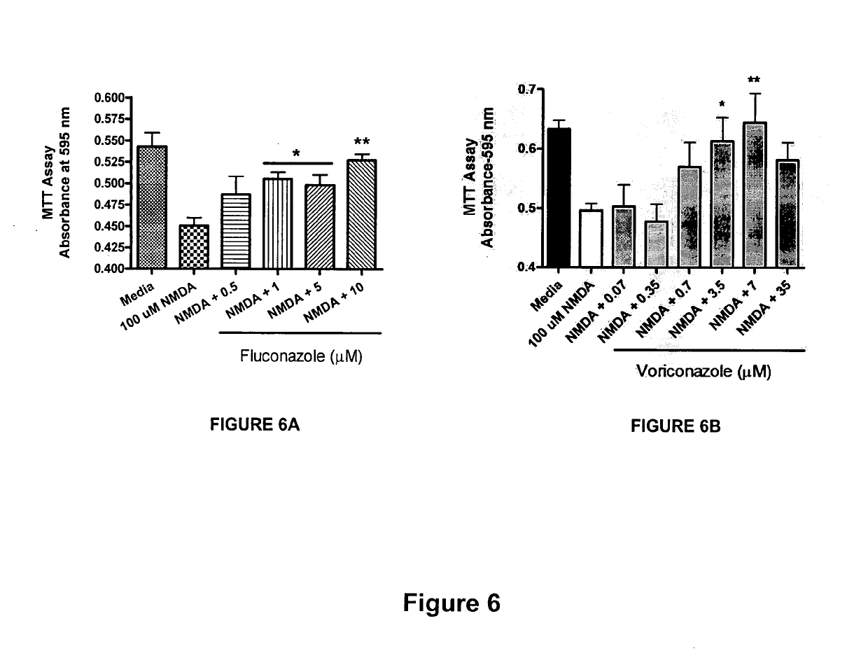

FIG. 6 illustrates the protection of hippocampal cultures from N-methyl-D-Aspartic Acid (NMDA) excitotoxicity with fluconazole and voriconazole (FIGS. 6A-6B, respectively). The neural and glial cells were preincubated with the neuroprotective compound for 1 hour followed by exposure to 100 μM of NMDA for 18 hours.

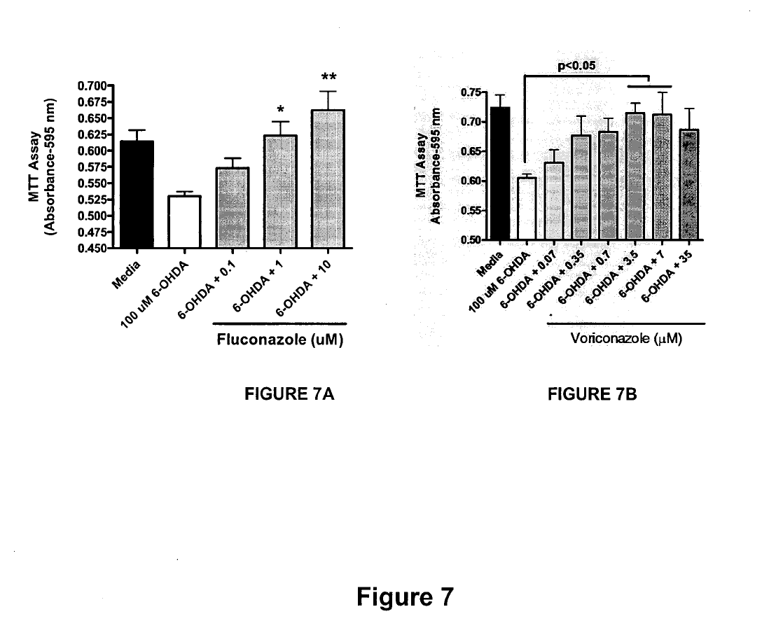

FIG. 7 illustrates the protection of hippocampal cultures from 6-OHDA with fluconazole and voriconazole (FIGS. 7A-7B, respectively). The neural and glial cells were preincubated with the neuroprotective compound for 1 hour followed by exposure to 100 μM of 6-OHDA for 18 hours.

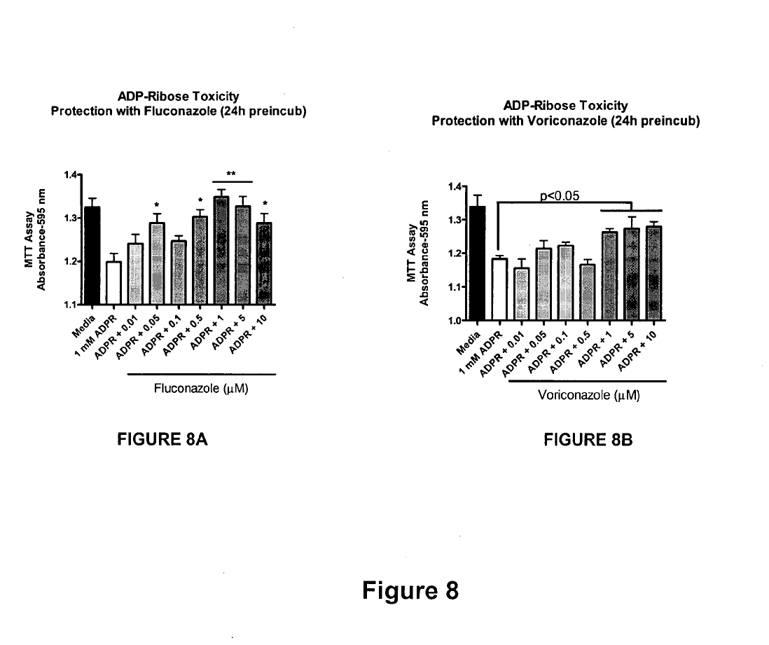

FIG. 8 illustrates the protection of hippocampal cultures from ADPRibose with fluconazole and voriconazole (FIGS. 8A-8B, respectively). The neural and glial cells were preincubated with the neuroprotective compound for 24 hours followed by exposure to 1 mM of ADPRibose for 18 hours.

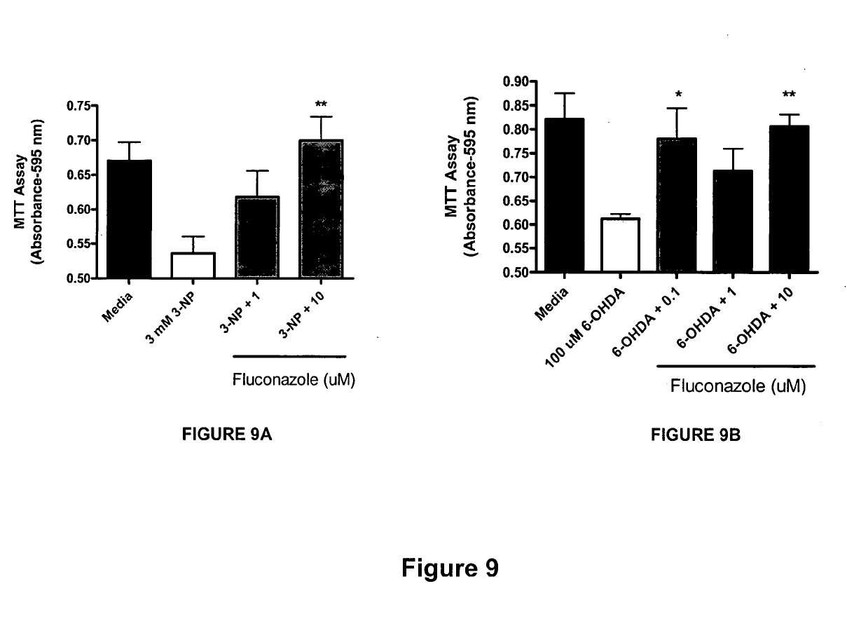

FIG. 9 illustrates the protection of human neuronal cultures from 3-NP and 6-OHDA with fluconazole (FIGS. 9A-9B, respectively). The neural and glial cells were preincubated with the neuroprotective compound for 1 hour followed by exposure to 3 mM 3-NP or 100 μM 6-OHDA for 18 hours.

Thus, the compounds disclosed herein demostrant broad range neuroprotection against a variety of neurotoxins.

Data from 8 replicates were utilized for each treatment group and were evaluated by ANOVA for significance. *p<0.05, **p<0.01, and ***p<0.001 from Newman Keuls pairwise comparisons to 3-NP, Tat and/or NMDA.

TABLE 2
Antifungal Agents Having Neuroprotective Character

| Compounds | Protection vs 3NP |
|---|---|
| KETOCONAZOLE | 20.93% |
| CLOTRIMAZOLE | 12.57% |
| EXALAMIDE | 9.19% |
| GRISEOFULVIN ANALOG B | 10.80% |
| FLUCONAZOLE | 13.35% |
| SULCONAZOLE NITRATE | 18.21% |
| GRISEOFULVIN ANALOG A | 22.47% |
| FLUTRIMAZOLE | 46.85% |
| TOLNAFTATE | 66.83% |
| ECONAZOLE NITRATE | 141.18% |
| TRIACETIN | 45.14% |
| MICONAZOLE NITRATE | 81.61% |
| RHAPONTIN | 22.12% |
| NYSTATIN | 46.72% |

TABLE 3
Antifungal Agents Having Neuroprotective Character

| Name | Structure | % Protection vs. 3-NP |
|---|---|---|
| Sulconazole Nitrate | 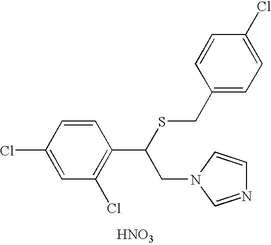 | 18.21 |
| Econazole Nitrate | 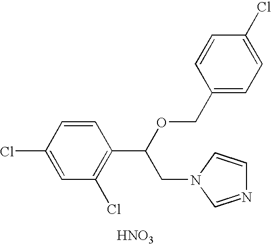 | 141.2 |
| Fluconazole | 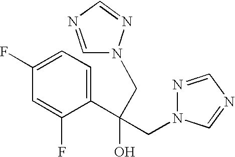 | 13.3 |
| Clotrimazole | 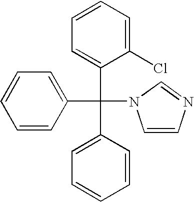 | 12.6 |
| Miconazole Nitrate | 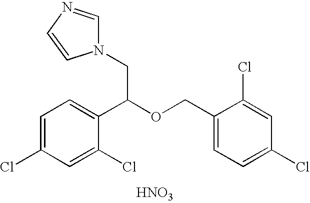 | 81.6 |
| Tolnaftate | 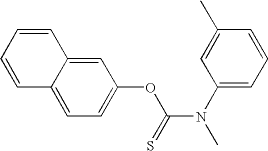 | 66.8 |

### Example 3B

### In Vitro Modeling of Blood Brain Barrier Permeability to Test Compounds

In vitro models of the Blood Brain Barrier from human brain microvascular endothelial cells (HBMEC) that were isolated and characterized have been published previously (see, e.g., Stins et al. (1997), J Neuroimmunol, 76(1-2):81-90; Cucullo et al. (2007), Epilepsia, 48(3):505-5l6). These HBMEC possess gamma glutamyl transpeptidase (GGTP) and drug transporter P-glycoprotein, and junctional proteins as seen by ZO1 immuno-staining, thereby demonstrating their brain endothelial cell characteristics. In vitro BBB models are constructed by growing HBMEC on microporous membranes (0.4 μm pore size) in the upper compartment of semipermeable Transwell™ tissue culture inserts (24 wells, Corning-Costar). The upper compartment compares to blood side and bottom compartment to brain side. Transmission electron microscopy reveals a smooth endothelial cell monolayer, typical rod shaped Weibel-Palade bodies and tight junctions. Polarity is shown after treatment with TNF-α, which results in an apical expression of ICAM-1. The presence of junctional proteins is seen by ZO1 immuno-staining and Western blotting for ZO-1, beta-catenin and occludin-1, showing that HBMEC possess endothelial and brain characteristics and functions.

Propidium iodide (PI) (MW=600) at 0.5 mg/ml, an indicator of in vitro blood brain barrier (“BBB”) integrity, is applied to the upper compartment along with a vehicle solution or a solution containing test compound at a concentration of 1 μM. At 2 and 4 hours post drug treatment, levels of compound are measured in top and bottom compartments by mass spectrometery as described in Tian et al. (2004), Rapid Comm Mass Spec, 18:3099-3104. The level of test compound detected in the bottom compartment is then normalized for differences in PI permeability between the test compound solution and the vehicle control solution.

### Example 3C

### In Vivo Assessment of Neuroprotective Compounds in a 3-NP-Induced Neurotoxicity Animal Model

In order to determine the in vivo efficacy of compounds identified as neuroprotective against 3-NP in vitro, as described herein, employed is a 3-NP-induced neurotoxicity model in rats. See, e.g., Kumar et al. (2006), Behav Pharmacol, 17(5-6):485-492.

To induce 3-NP neurotoxicity in vivo, 12-week-old male Lewis rats weighing 340-370 gm are administered intraperitoneal injections of 3-NP (20 mg/kg) for 4 days. The animals are divided into three treatment groups (n=12 per group) as follows:

Group 1 is administered fluconazole, miconazole, clomitrazole, econazole, tolnaftate, or another neuroprotective compounds disclosed herein once daily (10 mg/kg; oral gavage) beginning four days prior to and continuing for four days subsequent to the beginning of the 3-NP injections.

Group 2 is administered saline vehicle (negative control), by oral gavage, beginning four days prior to and continuing for four days subsequent to the beginning of the 3-NP injections.

Group 3 is administered resveratrol (positive control) (10 mg/kg; oral gavage) by oral gavage, beginning four days prior to and continuing for four days subsequent to the beginning of the 3-NP injections.

Subsequent to the beginning of the 3-NP injections, animals are assessed for significant loss of body weight, a decline in motor function (locomotor activity, movement pattern, and vacuous chewing movements) and cognitive deficits (e.g., impairment in learning or memory). Differences of performance between groups are analyzed at each time point by two-tailed t test.

Twenty-four hours after the last 3-NP injection animals are anesthetized, perfused transcardially with saline, followed by ice-cold 4% paraformaldehyde in 0.1 M phosphate buffer, pH 7.4. Brains are immediately removed and postfixed overnight in the same fixative and then cryoprotected in 30% sucrose in 0.1 M phosphate buffer, pH 7.4. Sequential coronal sections (30 μm) are made on a freezing microtome, starting from the anterior aspect of the corpus callosum throughout the entire striatum. For histological assessment, every sixth section (210 μm interval) is processed for cresyl violet staining to assess cell loss and neuronal degeneration. Cresyl violet staining is performed with standard protocols.

Stereological analysis of lesion volumes are performed by digitally acquiring cresyl violet-stained sections through the striatum at 4× objective using a computerized image analysis system. Lesion volumes for each group of animals are calculated by summing the cross-sectional areas of the lesion in each section and multiplying this value by the distance between sections.

Compounds found to confer a significant reduction in 3-NP-induced behavioral deficits or neuroanatomical lesion volume are considered to be neuroprotective in vivo.

### Example 3D

### In Vivo Assessment of Identified Neuroprotective Compounds in a Focal Ischemia Animal Model

A focal lesion model induces a localized inflammatory response and subsequent lesion only within a specific region of the spinal cord. The focal lesion model therefore enables an accurate assessment of the efficacy of neuroprotective agents because a specific region of the spinal cord and brain are compared between animals. Furthermore, the focal lesion model decreases variability found within the classic EAE models and therefore decreases the number of animals required.

Lewis rats (12-15 rats per treatment group) are treated with a test compound at a dose of 0.1, 1, and 10 mg/kg s.c or p.o. or with vehicle once daily for 3 days prior to immunization with MOG and incomplete Freunds Adjuvant as described below. Eighteen days later, the animals are subjected to laminectomy of the dorsal column at T8 and injected with TNF-α and γ-IFN to induce a focal EAE lesion as described below. Three weeks later, the animals are sacrificed. Outcome measures include behavioral studies (Basso-Beattie-Bresnahan scale), evoked potentials (nerve conduction velocity), radiological outcomes (Diffusion Tensor Imaging, Magnetic Resonance Imaging), and histology measurements (Luxol Fast Blue, Toluidine Blue, Myelin Basic Protein, phosphoNeurofilament and Axonal degeneration). A second cohort includes another set of animals where the same drug regimen listed above is initiated following the laminectomy.

### Animals

For these experiments, Lewis rats (10-12 weeks old) are allowed free access to food and water to acclimate 7-10 days before the initiation of experiments. At the time of the study, the animals weigh 200-250 g.

### Induction of Myelin Oligodendrocyte Glycoprotein (MOG) Sensitivity

Animals are injected subcutaneously at the base of the tail with 100 μl of recombinant MOG₁₋₁₂₅ (250-500 μg/mL) emulsified in incomplete Freund's adjuvant. No manipulations are performed for 18-30 days after immunization to allow for the immune system to develop a sensitivity to MOG. This procedure does not induce clinical symptoms of EAE.

### Induction of Focal EAE Lesion

To induce an EAE lesion within a specific region of the spinal cord, a steriotaxic injection of TNF-α and IFN-γ is administered within the spinal cord after laminectomy. The animals are shaved prior to initiation of the procedure. Anesthetized animals are placed in the prone position, and a midline incision is made. Following the dissection of fascia and muscle, a laminectomy is performed at T8 with Rongeur forceps. A focal EAE lesion within the cortical spinal tract is induced by administering 2 μL of 250 ng of TNF-α and 150 Units of IFN-γ dissolved in phosphate buffered saline with trace amounts of Monastral Blue using a capillary glass tube. Following surgery the wound is sutured first through the fascia, and then the skin with a 4.0 vicryl thread or with wound clips. Animals are warmed and allowed to recover.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a neurodegenerative disorder, peripheral neuropathy, or neuropathic pain comprising administration to an individual in need thereof a therapeutically effective amount of a compound having the formula: 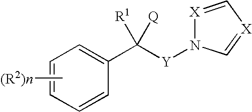
wherein,
each X is independently CH and N, and wherein at least one X is N;
Y is a bond or —CR′R″—;
R′ and R″ are independently selected from hydrogen, substituted or unsubstituted alkyl, substituted or unsubstituted alkoxy, and halo; or
R′ and R″, when taken together, are oxo;
Q is H or —C(R³)₃;
n is 5;
R¹ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
R¹ and Q are taken together to form ═N—R⁵, R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl;
R² is selected from hydrogen, halo, —OR⁴, —S12⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl;
each R³ is independently selected from hydrogen, halo, —OR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
a pair of R³ groups are taken together to form a heterocycloalkyl or cycloalkyl group; or
three R³ groups are taken together to form a substituted or unsubstituted aryl group or a substituted or unsubstituted heteroaryl group; or
R⁴ and an R³ taken together are —(C(R⁶)₂)ₘ—;
each R⁶ is independently selected from hydrogen, halo, -alkyl-O—R⁷, -alkyl-S—R⁷, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl; m is 1-5;
each R⁷ is independently selected from hydrogen, substituted or unsubstituted alkyl, substituted or unsubstituted aryl, substituted or unsubstituted heteroaryl, substituted or unsubstituted cycloalky, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-arylheterocycloalkyl-aryl, and substituted or unsubstituted heteroaryl-aryl-heterocycloalkyl-aryl;
each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl or a pharmaceutically acceptable salt thereof.

2. The method of claim 1, wherein the compound is selected from sulconazole, econazole, clotrimazole, miconazole, bifonazole, fenticonizole, isoconazole, oxiconazole, sertaconazole, tioconazole, fluconazole, butoconazole, isavuconazole, ravuconazole, voriconazole, albaconazole, terconazole, posaconazole, or a pharmaceutically acceptable salt, steroisomer, tautomer, or solvate thereof.

3. The method of claim 2, wherein the pharmaceutically acceptable salt thereof is a nitrate.

4. The method of claim 1, wherein the neurodegenerative disorder is a neurodegenerative disease selected from Alzheimer's Disease, Multiple Sclerosis, HIV-associated dementia, Huntington's Disease, Parkinson's Disease, and Amyotrophic Lateral Sclerosis.

5. The method of claim 2, wherein the neurodegenerative disease is selected from Multiple Sclerosis, HIV-associated dementia, Huntington's Disease, Parkinson's Disease, and Amyotrophic Lateral Sclerosis.

6. The method of claim 1, wherein the neurodegenerative disorder is a neurodegenerative condition selected from stroke and ischemia.

7. The method of claim 1, wherein the compound is an inhibitor of fungal ergosterol biosynthesis.

8. The method of claim 7, wherein the compound is an inhibitor of lanosterol-14a-demethylase (CYP51).

9. The method of claim 1, wherein the method is a method of treating a neurodegenerative condition and the compound is administered systemically.

10. The method of claim 9, wherein the compound is blood-brain barrier penetrating.

11. The method of claim 10, wherein the compound is selected from fluconazole, voriconazole, posaconazole and ravuconazole.

12. The method of claim 1, wherein the method is a method of treating peripheral neuropathy or systemic pain and the compound is administered locally.

13. (canceled)

14. The method of claim 1, wherein the compound blocks TRPM2 channel signaling.

15. A method of reducing neuronal or glial cell death by contacting a plurality of neurons or glial cells in need of protection from cell death with an effective amount of a compound having the formula: 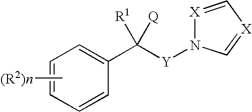
wherein,
each X is independently CH and N, and wherein at least one X is N;
Y is a bond or —CR′R″—;
R′ and R″ are independently selected from hydrogen, substituted or unsubstituted alkyl, substituted or unsubstituted alkoxy, and halo; or
R′ and R″, when taken together, are oxo;
Q is H or —C(R³)₃;
n is 5;
R¹ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
R² and Q are taken together to form R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl;
R² is selected from hydrogen, halo, —OR⁴, —SR⁴, —N(R⁴)₂, substituted or unsubstituted alkyl, substituted or unsubstituted aryl, substituted or unsubstituted hetcroaryl, substituted or unsubstituted cycloalkyl, substituted or unsubstituted heterocycloalkyl;
each R³ is independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
a pair of R³ groups are taken together to form a heterocycloalkyl or cycloalkyl group; or
three R³ groups are taken together to form a substituted or unsubstituted aryl group or a substituted or unsubstituted heteroaryl group; or
R⁴ and an R³ taken together are —(C(R⁶)₂)ₘ—;
each R⁶ is independently selected from hydrogen, halo, -alkyl-O—R⁷, -alkyl-S—R⁷, —SR⁴, —N(R⁴)₂,
substituted or unsubstituted alkyl, substituted or unsubstituted aryl, substituted or unsubstituted heteroaryl, substituted or unsubstituted cycloalkyl, and substituted or unsubstituted heterocycloalkyl;
m is 1-5;
each R⁷ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-arylheterocycloalkyl-aryl, and substituted or unsubstituted heteroaryl-aryl-heterocycloalkyl-aryl;
each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl
or a pharmaceutically acceptable salt thereof.

16. (canceled)

17. (canceled)

18. The method of claim 15, wherein the plurality of neurons are in a patient diagnosed with a neurodegenerative disorder, peripheral neuropathy or neuropathic pain.

19. The method of claim 18, wherein the neurological disorder is selected from Alzheimer's Disease, Multiple Sclerosis, HIV-associated dementia, Huntington's Disease, Parkinson's Disease, Amyotrophic Lateral Sclerosis, stroke and ischemia.

20. The method of claim 19, wherein the compound is an inhibitor of fungal ergosterol biosynthesis.

21. A pharmaceutical composition comprising a therapeutically effective amount of a compound having the formula: 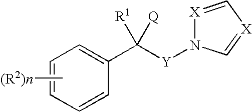
wherein,
each X is independently CH and N, and wherein at least one X is N;
Y is a bond or —CR^{′}R″—;
R′ and R″ are independently selected from hydrogen, substituted or unsubstituted alkyl, substituted or unsubstituted alkoxy, and halo; or
R′ and R″, when taken together, are oxo;
Q is H or —C(R³)₃;
n is 5;
R¹ is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
R¹ and Q are taken together to form ═N—R⁵, R⁵ is selected from OR⁴, substituted or unsubstituted alkyl, substituted or unsubstituted aryl, substituted or unsubstituted heteroaryl, substituted or unsubstituted cycloalkyl, substituted or unsubstituted heterocycloalkyl;
R² is selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl;
each R³ is independently selected from hydrogen, halo, —OR⁴, —SR⁴, —N(R⁴)₂, substituted or unsubstituted alkyl, substituted or unsubstituted aryl, substituted or unsubstituted heteroaryl, substituted or unsubstituted cycloalkyl, substituted or unsubstituted heterocycloalkyl; or
a pair of R³ groups are taken together to form a heterocycloalkyl or cycloalkyl group; or
three R³ groups are taken together to form a substituted or unsubstituted aryl group or a substituted or unsubstituted heteroaryl group; or
R⁴ and an R³ taken together are —(C(R⁶)₂)ₘ—;
each R⁶ is independently selected from hydrogen, halo, -alkyl-O—R⁷, —OR⁴, —N(R⁴)₂, substituted or unsubstituted alkyl, substituted or unsubstituted aryl, substituted or unsubstituted heteroaryl, substituted or unsubstituted cycloalkyl, and substituted or unsubstituted heterocycloalkyl;
m is 1-5;
each R⁷ is independently selected from hydrogen, substituted or unsubstituted alkyl, substituted or unsubstituted aryl, substituted or unsubstituted heteroaryl, substituted or unsubstituted cycloalkyl, substituted or unsubstituted heterocycloalkyl, substituted or unsubstituted arylheteroaryl, substituted or unsubstituted heteroarylaryl, and substituted or unsubstituted arylheterocycloalkyl, substituted or unsubstituted heterocycloalkylaryl, substituted or unsubstituted heterocycloalkyl-aryl-heterocycloalkyl-aryl, and substituted or unsubstituted heteroaryl-aryl-heterocycloalkyl-aryl;
each R⁴ is independently selected from hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted heterocycloalkylalkyl, substituted or unsubstituted cycloalkylalkyl, substituted or unsubstituted arylheterooaryl, and substituted or unsubstituted heteroarylaryl or a pharmaceutically acceptable salt thereof,
wherein the therapeutically effective amount is an amount sufficient to reduced neuronal cell death.

22. (canceled)

23. (canceled)

24. The composition of claim 21 further comprising a therapeutic agent for treating a neurodegenerative disorder.

25. The composition of claim 24, wherein the compound is fluconazole and the therapeutic agent is paroxetine.
